(19) United States
(12) Patent Application Publication
(10) Pub. No.: US 2025/0331763 A1
(43) Pub. Date: Oct. 30, 2025

(54) METHODS AND SYSTEMS FOR MEASURING EVOKED NEURAL RESPONSES

(71) Applicant: Saluda Medical Pty Limited, Macquarie Park, New South Wales (AU)

(72) Inventors: Zubin Zarir Nanavati, Macquarie Park (AU); Peter Scott Vallack Single, Macquarie Park (AU)

(21) Appl. No.: 19/177,163

(22) Filed: Apr. 11, 2025

(30) Foreign Application Priority Data
Apr. 12, 2024 (AU) 2024901027

(51) Int. Cl. A61B 5/388; A61B 5/294; A61B 5/302; A61B 5/311

(52) U.S. Cl. CPC A61B 5/388; A61B 5/294; A61B 5/302; A61B 5/311

(57) ABSTRACT

Disclosed is an implantable device for measuring an evoked neural response. The implantable device comprises a stimulus source configured to deliver neural stimuli via one or more stimulus electrodes to neural tissue, the neural stimuli being configured to evoke a neural response from the neural tissue. The implantable device further comprises a measurement amplifier configured to amplify a signal sensed between a first input of the measurement amplifier by a first measurement electrode and a second input of the measurement amplifier by a second measurement electrode subsequent to a provided neural stimulus, the sensed signal comprising the evoked neural response. The implantable device further comprises a control unit configured to: control the stimulus source to deliver a neural stimulus; and measure the evoked neural response of the amplified sensed signal. The implantable device further comprises one or more impedance elements configured to provide a negative impedance to at least one of the first and second inputs of the measurement amplifier.

The present application claims priority from Australian Provisional Patent Application No 2024901027 filed on 12 Apr. 2024, the contents of which are incorporated herein by reference in their entirety.

### TECHNICAL FIELD

The present invention relates to measurement of neural responses, such as compound action potentials, evoked by neurostimulation, and in particular to reducing or eliminating artefact generated by measurement circuitry in response to transient effects induced by an applied neural stimulus.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, movement disorders, and voiding disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal). Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-cord distance and thus the recruitment efficacy of a given stimulus. Moreover, the spinal cord itself moves within the cerebrospinal fluid (CSF) with respect to the dura. During postural changes, the amount of CSF or the distance between the spinal cord and the electrode can change significantly. This effect is so large that postural changes alone can cause a previously comfortable and effective stimulus regime to become either ineffectual or painful.

Another control problem facing neuromodulation devices of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable devices, increased power consumption results in more frequent charging and, given that batteries only permit a limited number of charging cycles, this ultimately reduces the implanted lifetime of the device.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli so as to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in electrode artefact, which may manifest in the sensed signal as a decaying output of the order of several millivolts after the end of the stimulus. As the neural response can be contemporaneous with the stimulus crosstalk or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result in a time-decaying artefact waveform of positive or negative polarity, their identification and elimination can be laborious.

Evoked neural responses are less difficult to measure when they appear later in time than the artefact, or when the signal-to-noise ratio is sufficiently high. The artefact is often restricted to a time of 1-2 ms after the stimulus and so, provided the neural response is measured after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

The difficulty of this problem is further exacerbated when attempting to implement CAP detection in an implanted device. Typical implanted devices have a power budget that permits a limited number, for example in the hundreds or low thousands, of processor instructions per stimulus, in order to maintain a desired battery lifetime. Accordingly, if a CAP detector for an implanted device is to be used regularly (e.g. once a second), then care must be taken that the detector should consume only a small fraction of the power budget.

A functional feedback loop can also produce useful data for live operation or post-analysis, such as observed neural response intensity and applied stimulus intensity. However, device operation at tens of Hz over the course of hours or days quickly produces large volumes of such data which far exceed an implanted device's data storage capacities.

Any discussion of documents, acts, materials, devices, articles or the like which has been included in the present Background is solely for the purpose of providing a context for the present invention. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide neural response measurement circuitry which will overcome or substantially ameliorate at least some of the deficiencies of the prior art, or at least provide an alternative.

According to a first aspect of the present technology, there is provided an implantable device for measuring an evoked neural response, the implantable device comprising: a stimulus source configured to deliver neural stimuli via one or more stimulus electrodes to neural tissue, the neural stimuli being configured to evoke a neural response from the neural tissue; a measurement amplifier configured to amplify a signal sensed between a first input of the measurement amplifier by a first measurement electrode and a second input of the measurement amplifier by a second measurement electrode subsequent to a provided neural stimulus, the sensed signal comprising the evoked neural response; a control unit configured to: control the stimulus source to deliver a neural stimulus; and measure the evoked neural response of the amplified sensed signal; and one or more impedance elements configured to provide a negative impedance to at least one of the first and second inputs of the measurement amplifier.

In some examples, the one or more impedance elements comprise: a first set of impedance elements connected to the first input of the measurement amplifier and the first measurement electrode; and a second set of impedance elements connected to the second input of the measurement amplifier and the second measurement electrode.

In some examples, the first and second sets of impedance elements provide respective first and second negative impedances to the first and second inputs in parallel to respective input impedances of the measurement amplifier at the respective inputs.

In some examples, the value of the negative impedance provided to each of the first and second inputs of the measurement amplifier is equal, or substantially equal, to the negative of the value of the input impedance of the measurement amplifier at the corresponding input.

In some examples, the value of the negative impedance provided to each of the first and second inputs of the measurement amplifier increases the value of a total impedance at the corresponding input of the measurement amplifier to at least a threshold impedance value.

In some examples, the threshold impedance value is sufficiently large to suppress a transient voltage generated at an electrode-tissue interface of a corresponding one of the measurement electrodes as a result of the neural stimulus to a degree that enables measurement of the evoked neural response.

In some examples, the one or more impedance elements comprise one or more respective negative impedance generator circuits.

In some examples, each negative impedance generator circuit comprises a Miller amplifier having a Miller impedance element connected across its input and its output, wherein a gain of the Miller amplifier is such that an effective input impedance to ground at the input of the Miller amplifier provides the negative impedance.

In some examples, the input of each Miller amplifier is connected to one of the first and second measurement electrodes.

In some examples, the input of at least one Miller amplifier is connected to the neural tissue.

In some examples, the value of the negative impedance provided by each negative impedance generator circuit is adjustable by adjusting a gain of the corresponding Miller amplifier.

In some examples, the value of each Miller impedance element is set to approximate, or be equal to, a corresponding input impedance of the measurement amplifier.

In some examples, each Miller impedance element is substantially capacitive with the capacitance value equal to a total input capacitance to ground at the corresponding input of the measurement amplifier.

In some examples, the first input of the measurement amplifier is connected to a first negative impedance generator circuit and the second input of the measurement amplifier is connected to a second negative impedance generator circuit.

In some examples, the first and second negative impedance generator circuits are independent circuits.

In some examples, each negative impedance generator circuit comprises a Miller amplifier having a Miller impedance element connected across its input and its output, and wherein each Miller amplifier has a pole with a cutoff frequency less than a frequency of the neural tissue and the Miller impedance element.

In some examples, the first and second negative impedance generator circuits share a common Miller amplifier.

In some examples, the common Miller amplifier drives a star point of a plurality of impedances arranged in a star configuration across the first input and the second input of the measurement amplifier.

In some examples, each impedance of the plurality of impedances provides a separate Miller impedance element to the common Miller amplifier.

In some examples, the plurality of impedances comprises a plurality of filter capacitors each having a capacitance of at least 100 pF.

According to a second aspect of the present technology, there is provided a method for measuring an evoked neural response, the method comprising: delivering a neural stimulus via one or more stimulus electrodes to neural tissue, the neural stimulus being configured to evoke a neural response from the neural tissue, and the neural stimulus being delivered according to a stimulus intensity parameter; capturing a signal sensed on the neural tissue by a first measurement electrode and a second measurement electrode, the sensed signal comprising the evoked neural response; using a measurement amplifier to amplify the sensed signal, the measurement amplifier having a first input connected to the first measurement electrode and a second input connected to the second measurement electrode; and measuring the neural response evoked by the delivered neural stimulus, wherein at least one of the first input and the second input of the measurement amplifier are provided with a negative impedance.

In some examples, the method further comprises configuring one or more impedance elements to provide at least one of the first input and the second input of the measurement amplifier with a negative impedance.

In some examples, the negative impedance provided to the at least one of the first input and the second input of the measurement amplifier is provided in parallel to an input impedance of the measurement amplifier at the at least one input.

In some examples, configuring one or more impedance elements comprises setting the value of the negative impedance provided to the at least one of the first and second inputs of the measurement amplifier to be equal, or substantially equal, to the negative of the value of the input impedance of the measurement amplifier at the at least one input.

In some examples, configuring one or more impedance elements comprises setting the value of the negative impedance provided to the at least one of the first and second inputs of the measurement amplifier to increase the value of a total input impedance of the measurement amplifier at the at least one input to at least a threshold impedance value.

In some examples, the threshold impedance value is sufficiently large to suppress a transient voltage generated at an electrode-tissue interface of the at least one corresponding measurement electrode as a result of the neural stimulus to a degree that enables measurement of the evoked neural response.

In some examples, the one or more negative impedances are generated by one or more respective negative impedance generator circuits.

In some examples, each negative impedance generator circuit comprises a Miller amplifier having a Miller impedance element connected across its input and its output, wherein a gain of the Miller amplifier is such that the effective input impedance to ground at the input of the Miller amplifier provides the negative impedance.

In some examples, the method further comprises adjusting the value of the negative impedance provided by each negative impedance generator circuit by adjusting a gain of the corresponding Miller amplifier.

In some examples, the method further comprises setting the value of each Miller impedance element to approximate, or be equal to, a corresponding input impedance of the measurement amplifier.

In some examples, each Miller impedance element is substantially capacitive with the capacitance value equal to a total input capacitance to ground at the corresponding inputs of the measurement amplifier.

In some examples, the first input of the measurement amplifier is connected to a first negative impedance generator circuit and the second input of the measurement amplifier is connected to a second negative impedance generator circuit.

In some examples, the first and second negative impedance generator circuits are independent circuits.

In some examples, each negative impedance generator circuit comprises a Miller amplifier having a Miller impedance element connected across its input and its output, and wherein each Miller amplifier has a pole with a cutoff frequency less than a frequency of the neural tissue and the Miller impedance element.

In some examples, the first and second negative impedance generator circuits share a common Miller amplifier.

In some examples, the common Miller amplifier drives a star point of a plurality of impedances arranged in a star configuration across the first input and the second input of the measurement amplifier.

In some examples, each impedance of the plurality of impedances provides a separate Miller impedance element to the common Miller amplifier.

In some examples, the plurality of impedances comprises a plurality of filter capacitors each having a capacitance of at least 100 pF.

In some examples, the method further comprises: computing, from an intensity of the measured evoked neural response, a feedback variable; and completing a feedback loop by using the computed feedback variable to control the stimulus intensity parameter so as to maintain the feedback variable at a target value.

According to a third aspect of the present technology, there is provided a neural stimulation system comprising: a neural stimulation device for controllably delivering neural stimuli to neural tissue, the device comprising: a control unit configured to: control a stimulus source to deliver a neural stimulus via one or more stimulus electrodes to neural tissue, the neural stimulus being configured to evoke a neural response from the neural tissue; and use a measurement amplifier having first and second inputs connected to corresponding first and second measurement electrodes to amplify a signal sensed by the first and second measurement electrodes subsequent to the delivered neural stimulus, the sensed signal comprising the evoked neural response, wherein at least one of the first and second inputs of the measurement amplifier are provided with a negative impedance; and a processor configured to measure the neural response evoked by the delivered neural stimulus based on the amplified sensed signal.

The present technology has been developed primarily for use in or with neuromodulation of the spinal cord and will be described hereinafter mostly with reference to this application. However, it will be appreciated that the present technology is not limited to this particular field of use, and may be applied in other neuromodulation contexts, including but not limited to sacral nerve stimulation, pudendal nerve stimulation, deep brain stimulation, stimulation of other parts of the peripheral and central nervous system. It will further be appreciated that the present technology may be applied for treatment of conditions other than chronic pain, including but not limited to movement disorders, Crohn's disease, rheumatoid arthritis, diabetes, Reynaud's phenomenon, pelvic floor disorders, chronic inflammatory conditions, migraine, stroke, or depression.

### BRIEF DESCRIPTION OF THE DRAWINGS

Notwithstanding any other implementations which may fall within the scope of the present invention, one or more implementations of the present invention will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is an illustration of the stimulus pulses delivered by a stimulation program with four interleaved stimulation sets (stimsets);

FIG. 9 is a schematic illustrating elements and inputs of a closed-loop neural stimulation (CLNS) system with multiple stimsets;

FIG. 10a is a circuit diagram of an example configuration of stimulation and measurement circuitry for measurement of an ECAP, according to one implementation of the present technology;

FIG. 10b is a schematic diagram of an electrical model of the configuration of FIG. 10a for providing neural stimulation to neural tissue and for measuring an evoked neural response, according to one implementation of the present technology;

FIG. 10c is a graph illustrating a voltage induced over a constant phase element (CPE) in response to a voltage step applied to a substantially capacitive input impedance of a measurement amplifier;

FIG. 11a is a diagram of electrical circuitry for measuring an evoked neural response, according to one implementation of the present technology;

FIG. 11b is a diagram of an example circuit model in which a first set of impedance elements are connected to a first measurement amplifier input and a second set of impedance elements are connected to a second measurement amplifier input, according to one implementation of the present technology;

FIG. 12a is a circuit diagram of an impedance generator circuit comprising a Miller amplifier with a gain and a Miller impedance connected between input and the output terminals;

FIG. 12b is a circuit diagram of an impedance generator circuit that is electrically equivalent to the impedance generator circuit of FIG. 12a, where the equivalent impedances are a function of the gain and the Miller impedance;

FIG. 13 is a circuit diagram of an exemplary impedance generator circuit (also referred to as a “Z-generator”), according to one implementation of the present technology;

FIG. 14 is a block diagram of a circuit model for measurement of neural responses with a plurality of independent negative impedance generator circuits, according to one implementation of the present technology;

FIG. 15 is a block diagram of a circuit model that is equivalent to one half of the model of FIG. 14, showing the addition of the Z-generator to the positive input of the measurement amplifier, according to one implementation of the present technology;

FIG. 16a is a graph containing plots of voltage across the CPE representing a corresponding measurement electrode during a 1 ms simulation of the models of FIG. 10b and FIG. 15 under stimulation, according to one implementation of the present technology;

FIG. 16b is a graph containing the plots of the voltages of FIG. 16a commencing 50 μs after the end of the stimulation pulse;

FIG. 17 is a diagram of a circuit model of an impedance generator circuit where the resistance values of resistors are set to be equal, according to one implementation of the present technology;

FIG. 18a is a graph containing plots of voltage across a CPE representing a corresponding measurement electrode during a 1 ms simulation, where the pole of the Miller amplifier was set to 1 Hz, according to one implementation of the present technology;

FIG. 18b is a graph containing the voltage plots of FIG. 18a commencing 350 μs after the end of the stimulation pulse;

FIG. 19 is a graph of simulated CPE voltages as the pole frequency of the operational amplifier varies, according to one implementation of the present technology;

FIG. 20 is a graph of the gain required for a given Miller capacitance to generate a virtual negative capacitance of −100 pF, according to one implementation of the present technology;

FIG. 21a is a block diagram of a model of a common Z-generator circuit connected to the CPE, according to one implementation of the present technology;

FIG. 21b is a block diagram of a model of a common Z-generator circuit connected to the measurement amplifier input, according to one implementation of the present technology;

FIG. 21c is a block diagram of a model containing a common Z-generator circuit that is approximately electrically equivalent to the circuit models of FIGS. 21a and 21b, according to one implementation of the present technology;

FIG. 22 is a block diagram of the circuit model of FIG. 21b in which a common Miller amplifier drives a network of capacitors arranged in a star configuration across the inputs of the measurement amplifier, according to one implementation of the present technology;

FIG. 23 is a block diagram of a circuit model equivalent to the model of FIG. 22, showing the addition of the common Z-generator to the positive input of the measurement amplifier, according to one implementation of the present technology;

FIG. 24a is a graph containing plots of voltages over a CPE representing a corresponding measurement electrode of the circuit model of FIG. 23 during a 1 ms stimulation pulse, according to one implementation of the present technology;

FIG. 24b is a graph containing the voltage plots of FIG. 24a commencing 350 μs after the end of the stimulation pulse;

FIG. 25a is a diagram of an equivalent circuit of the independent Z-generator model with a differential potential source having value V_{d}, according to one implementation of the present technology;

FIG. 25b is a diagram of an equivalent circuit of the Miller amplifier model with a differential potential source having value V_{d}, according to one implementation of the present technology;

FIG. 26 is a graph containing plots of frequency responses of the independent Z-generator model (VCVS), the common Miller amplifier model (“Star point driver”), and the model without negative impedance, to a differential voltage signal V_{d}, according to one implementation of the present technology;

FIG. 27 is a circuit diagram of an alternative implementation of the star point driver configuration of FIG. 22;

FIG. 28 is a flow diagram of a method for measuring an evoked neural response, according to one implementation of the present technology;

FIG. 29a is a graph illustrating a first neural response measurement conducted during a simulation without the use of negative impedance at the measurement amplifier;

FIG. 29b is a graph illustrating a second neural response measurement conducted during the simulation of FIG. 29a with the use of negative impedance at the inputs of the measurement amplifier, according to one implementation of the present technology;

FIG. 30 is an illustration of a scope capture of a common mode potential and a voltage at the output of a Miller amplifier of a Z-generator circuit to device ground, according to one implementation of the present technology; and

FIG. 31 is a graph illustrating a third neural response measurement conducted during the simulation of FIG. 29a with a gain set so that the magnitude of the negative capacitance exceeded the input capacitance of the measurement system, according to one implementation of the present technology.

### DETAILED DESCRIPTION OF THE PRESENT TECHNOLOGY

### Devices and Systems for Neuromodulation

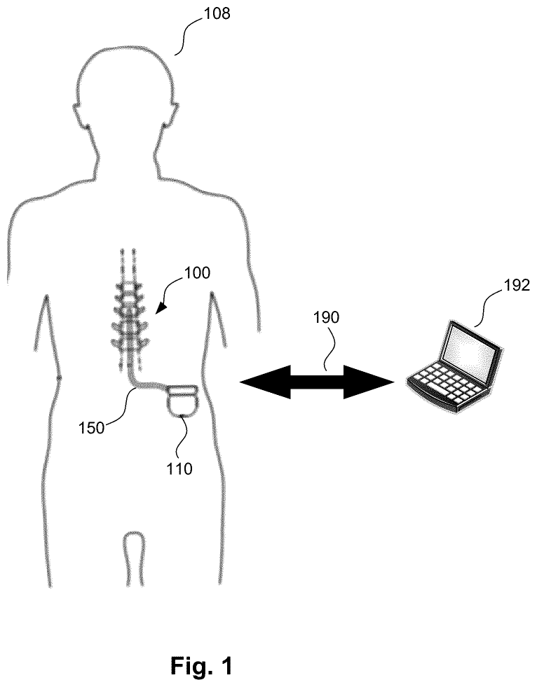

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly, typically comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

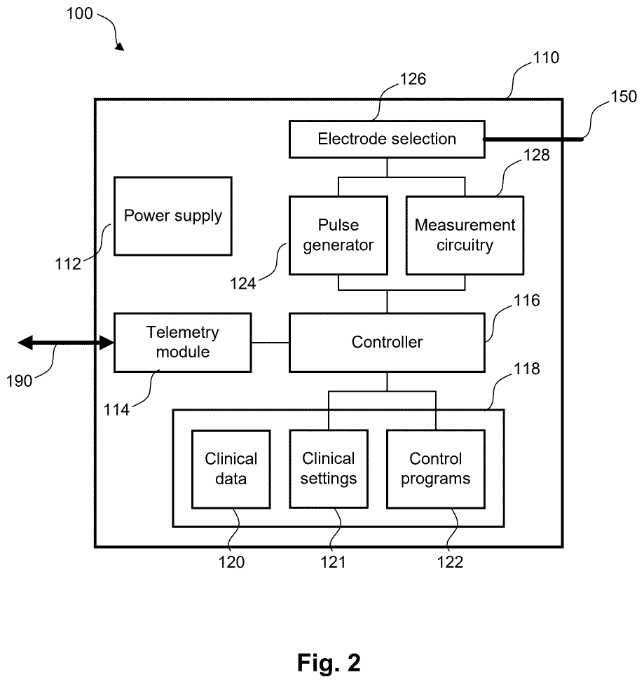

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode array 150 as selected by electrode selection module 126.

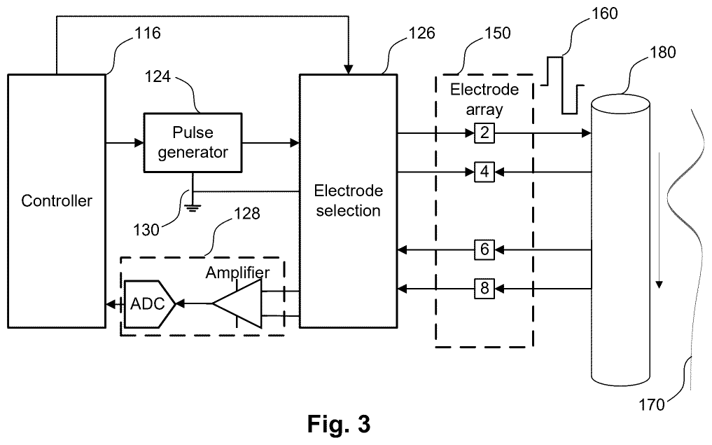

FIG. 3 is a schematic illustrating interaction of the implanted stimulator 100 with a bundle of target nerve fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve, or a brain structure. Electrode selection module 126 selects a stimulus electrode 2 of electrode array 150 through which to deliver a pulse from the pulse generator 124 to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be to create paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

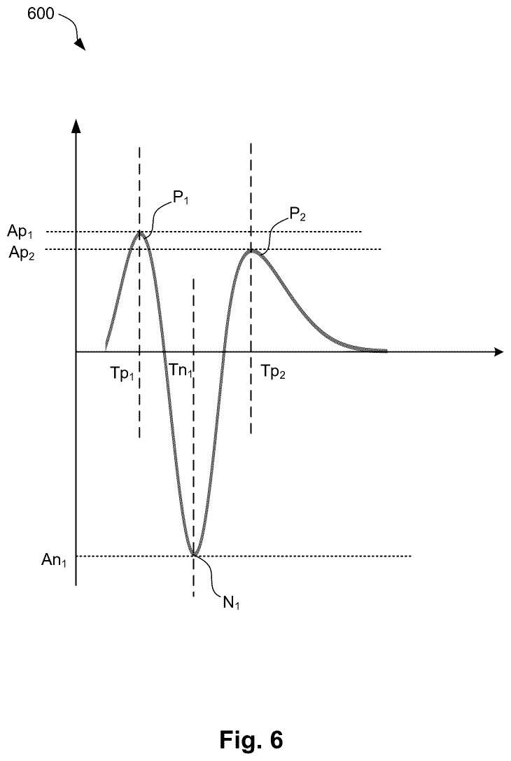

FIG. 6 illustrates the typical form of an ECAP 600 of a healthy subject, as sensed by a single measurement electrode referenced to the system ground 130 or to an indifferent electrode (a configuration referred to as single-ended ECAP measurement). The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode voltage fluctuations on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along target fibres 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, target response intensity, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

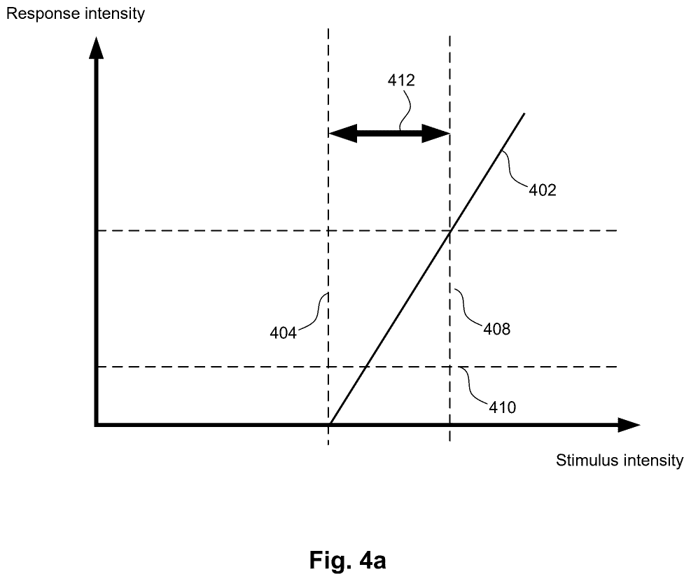

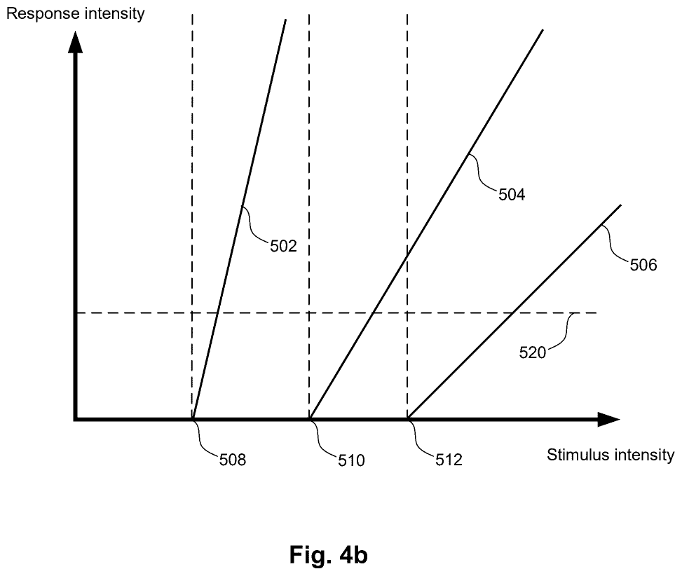

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$d = \{ \begin{matrix}S ( s - T ) , & s \geq T \\ 0 , & s < T\end{matrix} \qquad ( 1 )$$

where s is the stimulus intensity, d is the ECAP amplitude, T is the ECAP threshold and S is the slope of the activation plot (referred to herein as the patient sensitivity) above the ECAP threshold T. The sensitivity S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the spinal cord. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the patient sensitivity also changes, as indicated by the varying slopes of activation plots 502, 504, and 506. In general, as the distance between the stimulus electrodes and the spinal cord increases, the ECAP threshold increases and the sensitivity decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at or near a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at or near an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, the user sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

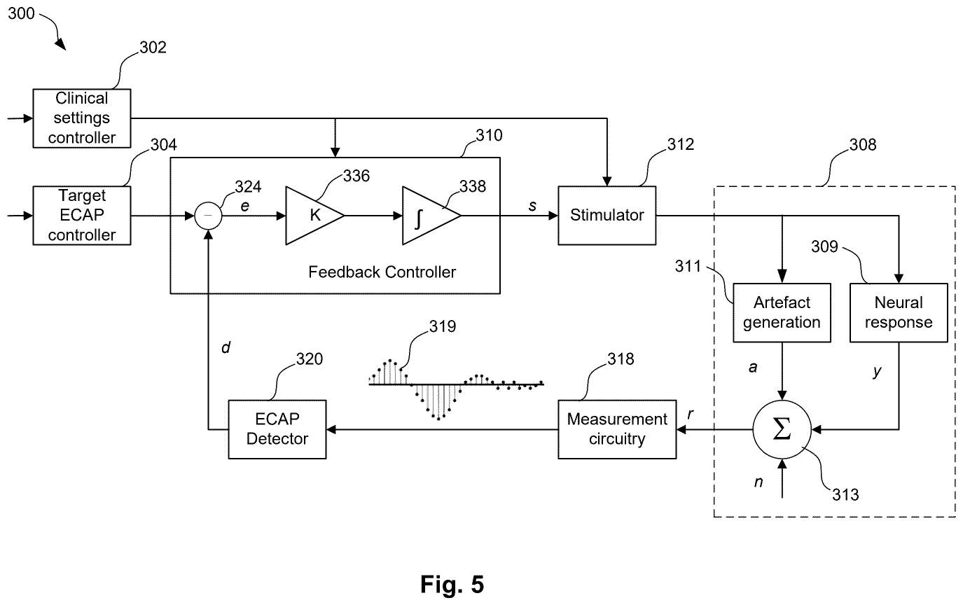

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal r to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int Kedt \qquad ( 1 )$$

where K is the gain of the gain element 336 (the controller gain). This relation may also be represented as

$$δ s = Ke \qquad ( 2 )$$

where δs is an adjustment to the current stimulus intensity parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

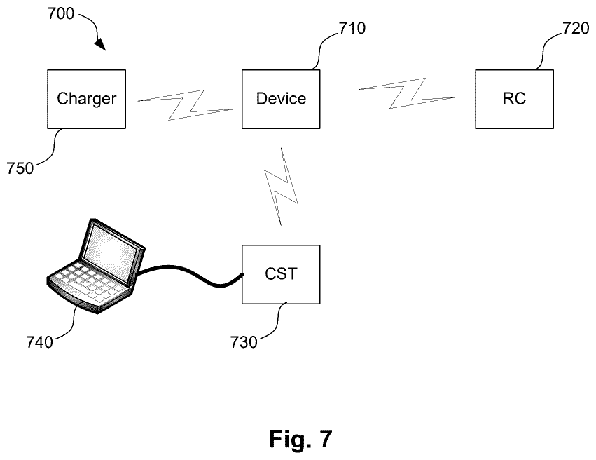

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

For some patients, it is beneficial for a neural stimulation therapy program to comprise multiple stimulation sets. A stimulation set (“stimset”) is a set of stimulus and return electrodes, or more precisely a stimulus electrode configuration (SEC), along with the stimulus parameters that govern the stimulation pulses delivered via that SEC.

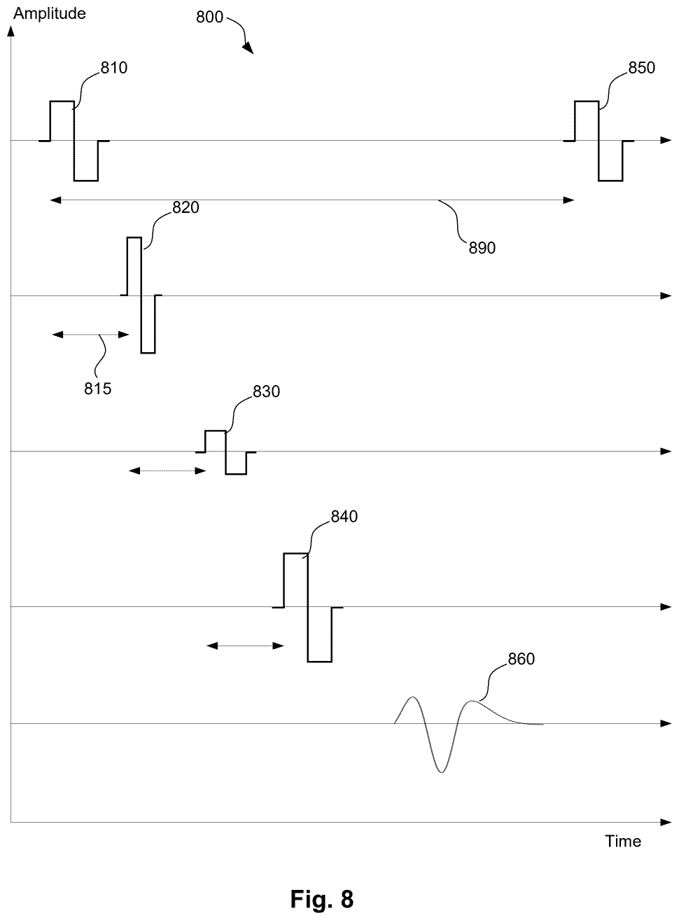

FIG. 8 is an illustration 800 of the stimulus pulses delivered by a stimulation program with four interleaved stimsets. The stimulus pulse train delivered according to each stimset is illustrated on a separate, but vertically aligned, horizontal axis representing time. All the stimulus pulse trains are delivered at the same stimulus frequency. (It is not a requirement that all the stimulus pulse trains for the respective stimsets are delivered at the same stimulus frequency; however it is so represented in FIG. 8 for ease of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 8 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter. The stimulus period 890 is the period of repetition of the full cycle and is equal to the reciprocal of the stimulus frequency. In one implementation, the ISI 815 is the stimulus period divided by the number of stimsets, so that the stimuli are evenly spaced throughout the stimulus period 890.

Also illustrated is an evoked neural response in the form of an evoked compound action potential (ECAP) 860 as sensed by a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 8, may be based on measurements of the ECAP 860. That is to say, closed-loop adjustments to the stimulus parameters of all stimsets may all be based on measurements of the ECAP 860 from a single stimset, referred to as the applied stimset. In FIG. 8, the final stimset in the cycle is the applied stimset.

If the ISI 815 is short, ECAPs evoked by the first three stimulus pulses 810, 820, and 830 are potentially obscured by stimulus crosstalk or artefact from the stimulus pulses 820, 830, and 840. Therefore, if the ISI 815 is short, only the final stimset in the cycle may evoke a measurable ECAP. If the ISI 815 is greater than the refractory period and sufficiently long that ECAPs evoked by the earlier stimsets are not obscured by stimulus crosstalk or artefact from the other stimulus pulses in the cycle, any of the stimsets in the cycle may evoke a measurable ECAP and may therefore be the applied stimset.

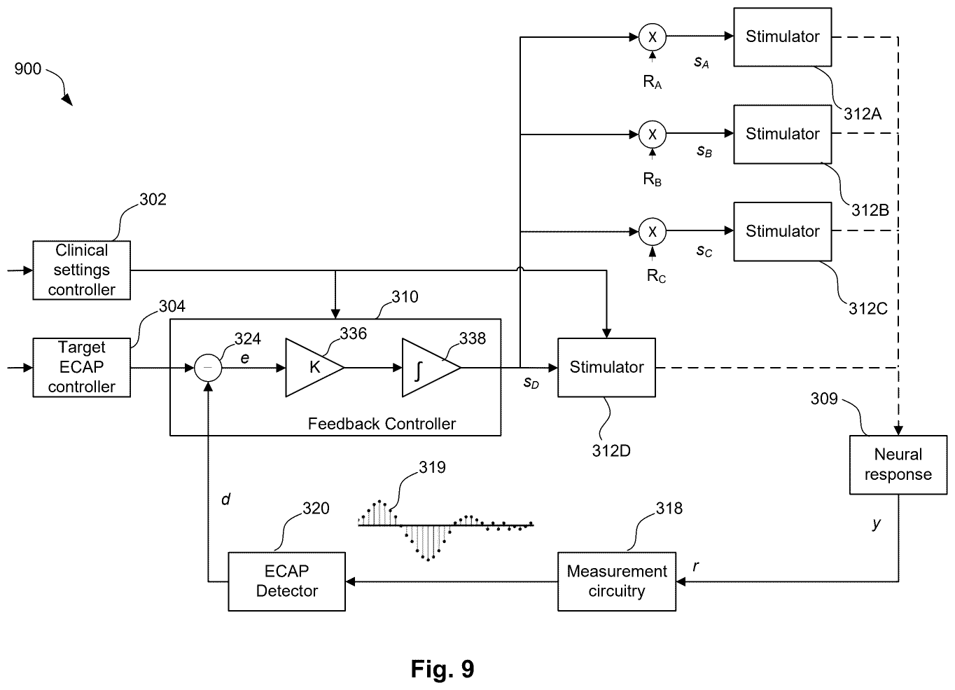

FIG. 9 is a schematic illustrating elements and inputs of a multi-stimset CLNS system 900 with multiple stimsets. The multi-stimset CLNS system 900 is the same as the CLNS system 300 of FIG. 5, with like numbers indicating like elements, with the addition of three further stimsets. The noise addition and artefact generation in FIG. 5 have been omitted from FIG. 9 for clarity. The four stimsets are labelled A, B, C, and D and are delivered by stimulators 312A, 312B, 312C, and 312D (the latter of which corresponds to the stimulator 312 in the CLNS system 300) according to respective stimulus intensity parameters s_{A}, s_{B}, s_{C}, and s_{D}, and via respective SECs. The pulses delivered by the stimulators 312A, 312B, 312C, and 312D correspond to the stimulus pulses 810, 820, 830, and 840 of FIG. 8. The neural response y may be measured from any of stimsets A, B, C, and D, which is why the neural response box 309 is joined by dashed lines to all four stimulators 312A, 312B, 312C, and 312D in FIG. 9. In the implementation of FIG. 8, the stimulus intensity parameter s_{D} for stimset D is the largest of the four stimulus intensity parameters s_{A}, s_{B}, s_{C} and s_{D} and is the stimulus intensity parameter that is directly adjusted by the feedback controller 310. The stimulus intensity parameter s_{D} is scaled by ratios R_{A}, R_{B}, and R_{C} to obtain the stimulus intensity parameters s_{A}, s_{B}, and s_{C} for stimsets A, B, and C respectively at the end of each cycle. The ratios R_{A}, R_{B}, and R_{C}, which are all less than or equal to one, are fixed at the ratios of the respective stimulus intensities s_{A}, s_{B}, and s_{C} at which the respective stimsets were originally programmed, to the originally programmed stimulus intensity s_{D} of the applied stimset D and form part of the clinical settings 121 of the multi-stimset program. In such an implementation, the stimulus intensity parameters s_{A}, s_{B}, and s_{C} always remain in fixed ratio with the stimulus intensity parameter s_{D} and with each other. This is referred to as ratiometric adjustment. So for example, if the originally programmed stimulus intensities were 1 mA, 2 mA, 4 mA, and 6 mA for the four stimsets A, B, C, and D respectively, the ratios R_{A}, R_{B}, and R_{C} are fixed at programming time at ⅙, ⅓, and ⅔ respectively. If during therapy the feedback controller 310 adjusts the largest stimset intensity parameter s_{D} to 6.6 mA, the stimulus intensity parameters s_{A}, s_{B}, and s_{C} are automatically adjusted to 1.1 mA, 2.2 mA, and 4.4 mA respectively. The clinical settings controller 302 provides to the stimulators 312A, 312B, 312C, and 312D the stimulus parameters that are not under the control of the feedback controller 310.

It may be seen from FIG. 9 that the adjustments to the stimulus intensity parameters after each stimulus cycle are all in fixed proportion. A ratiometric multi-stimset CLNS system therefore emulates a CLNS system with four separate feedback loops driven by the four stimsets, wherein each loop has the same controller gain. A ratiometric multi-stimset CLNS system is effective to maintain the responses evoked by each stimset at a constant neural response intensity on the condition that when the patient moves to a new posture, the threshold and slope of all activation plots, both for applied and non-applied stimsets, move in a proportional manner. (See FIG. 4b for examples of activation plots for a given stimset in different postures.)

### Mismatch Artefact in Neural Response Measurement

Recording an evoked neural response, such as an ECAP, requires the delivery of an electrical stimulus, by one or more stimulus electrodes, and the recording of electrical parameters of a signal produced by the stimulated neural pathway, as sensed by one or more measurement electrodes. This is challenging because stimulation results in crosstalk at the measurement electrodes, which can in turn induce artefact that is much larger than the evoked action potentials.

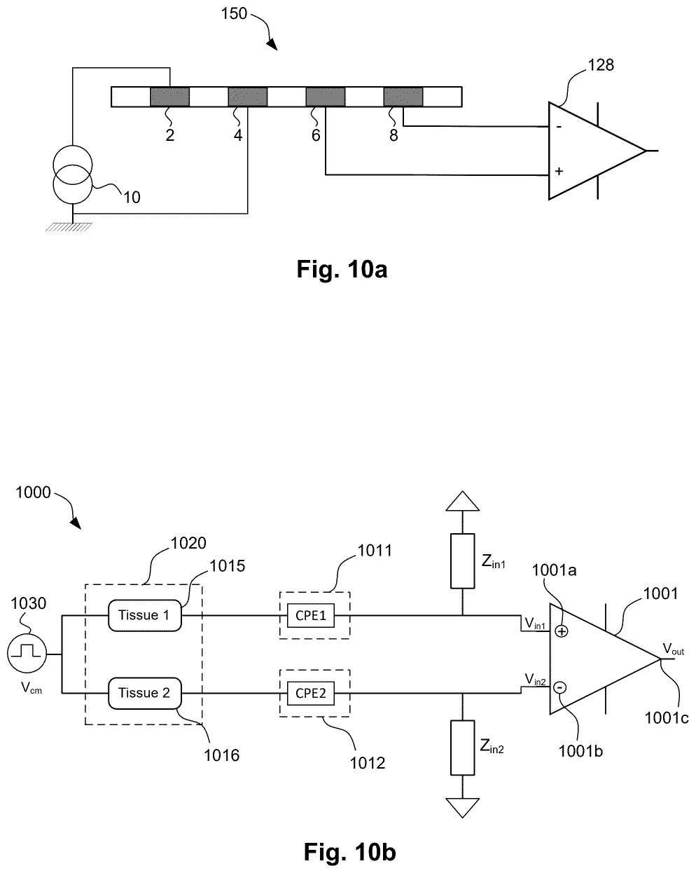

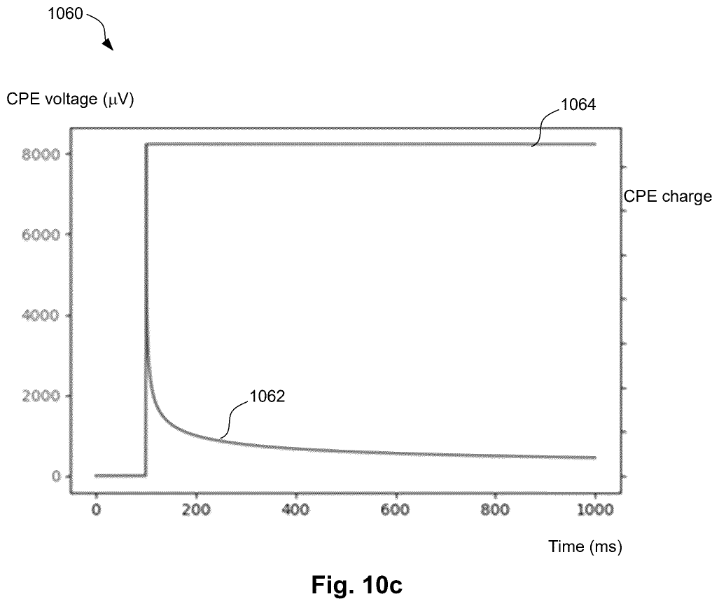

FIG. 10a illustrates an example configuration of stimulation and measurement circuitry, including stimulus and measurement electrodes, for measurement of an ECAP. This configuration utilises an implantable electrode array 150, as shown in FIG. 3, connected to a stimulus source 10 and comprising stimulus electrodes 2, 4, a measurement recording electrode 6, a measurement reference electrode 8, and measurement circuitry 128.

FIG. 10b illustrates an electrical model 1000 of the configuration of FIG. 10a for providing neural stimulation to neural tissue and for measuring an evoked neural response. The stimulus, modelled as a common mode potential (a voltage pulse) 1030 having an amplitude extending from ground (“GND”) to the supply voltage VDDHV, is delivered to neural tissue 1020 via the stimulus electrodes (not shown). The neural tissue 1020 may be modelled as an ionic solution which, in some examples, is electrically modelled by a resistor network. A first measurement electrode 1011 and a second measurement electrode 1012 are each connected to respective tissue components 1015 and 1016 of the neural tissue 1020. The electrode-tissue interfaces are metal-electrolyte interfaces modelled by a set of constant-phase elements (CPEs) denoted CPE1 and CPE2 for the first measurement electrode 1011 and the second measurement electrode 1012 respectively. The first and second measurement electrodes 1011 and 1012 are connected to measurement amplifier 1001 at a corresponding first input 1001a and a second input 1001b. Measurement amplifier 1001 is a differential amplifier configured to amplify a difference between the voltages at the first and second input terminals, denoted Vᵢₙ₁ and Vᵢₙ₂, which result from voltages delivered over the CPEs CPE1 and CPE2 of the first 1011 and second 1012 measurement electrodes.

Stimulus is delivered to the tissue components 1015 and 1016 resulting in the common mode potential 1030 inducing voltages Vᵢₙ₁ and Vᵢₙ₂ at the inputs 1001a and 1001b of amplifier 1001. Measurement amplifier 1001 outputs an output voltage Vₒᵤₜ that is proportional to the difference Vᵢₙ₁−Vᵢₙ₂. Ideally, any sensed difference in the input voltages Vᵢₙ₁ and Vᵢₙ₂ is due to the neural response, where amplification of the sensed signal by the measurement amplifier 1001 facilitates measurement of one or more parameters of the response (e.g., a response intensity).

In practice, measurement amplifier 1001 is non-ideal and has a finite input impedance. The input impedance of measurement amplifier 1001 at each of the first input 1001a and the second input 1001b is represented by corresponding impedance elements Zᵢₙ₁ and Zᵢₙ₂. During the delivery of the stimulus, currents will flow through the measurement electrodes 1011 and 1012 from the common mode potential 1030 imparted on the tissue components 1015 and 1016. The common mode potential 1030 varies from GND to VDDHV inducing a current through the elements CPE1 and CPE2 of the measurement electrodes 1011 and 1012 and the input impedance elements Zᵢₙ₁ and Zᵢₙ₂, and causing each element CPE1 and CPE2 to produce a voltage transient at the respective inputs 1001a and 1001b of the amplifier 1001. Further, neural tissue components 1015 and 1016 may experience a potential difference from the change in value of the common mode potential 1030 purely from the connection of the return electrode to VDDHV or GND independently of whether a stimulus current is being delivered to the neural tissue. That is, the connection of the neural tissue to VDDHV or GND causes currents to flow through CPE1 and CPE2 and into the non-infinite input impedances Zᵢₙ₁ and Zᵢₙ₂ of the measurement amplifier 1001.

The measurement amplifier 1001 produces output voltage Vₒᵤₜ that is proportional to the input voltage difference (Vᵢₙ₁−Vᵢₙ₂). In response to the first and second measurement electrodes having matched characteristics, the voltage transients produced at each input are equal and the measurement amplifier output will therefore be unaffected by the common mode potential 1030 (since Vᵢₙ₁−Vᵢₙ₂=0).

However, there is often a mismatch between the impedance values of the measurement electrodes 1011 and 1012, as represented by corresponding values of CPE1 and CPE2. The impedance mismatch can result from one or more sources. For example, tissue growth on the electrode contacts may cause a reduction in the surface area of the electrode-tissue interface resulting in an increase in the tissue component impedance or the CPE impedance. Alternatively, or in addition, variability in the surface finish of the first or second measurement electrode may be another source of CPE impedance mismatch.

The voltage transient produced by each CPE element is generally inversely proportional to the corresponding input impedances Zᵢₙ₁ and Zᵢₙ₂ of the measurement amplifier 1001. Several components contribute to the input impedance, including: the measurement amplifier itself, any RF filter network(s) connected to the input, inter-lead capacitances, and stray capacitances (such as PCB capacitance). For example, the input impedance of the measurement amplifier 1001 is substantially capacitive as a result of the inclusion of an RF filter network (e.g., of 100 pF) at the inputs 1001a and 1001b of the amplifier 1001. For such a substantially capacitive input impedance with a capacitance of C, a step change in the common mode potential of magnitude V at a tissue component subjects the corresponding CPE to a current impulse with an approximate charge content Q of:

$$Q = C V \qquad ( 4 )$$

FIG. 10c is a graph 1060 illustrating a voltage induced across a CPE in response to a voltage step applied to a substantially capacitive input impedance of a measurement amplifier. The CPE voltage trace 1062, as generated by a current impulse at t=100 μs with a charge content Q of 1.5 nC, is plotted against the waveform 1064 of delivered charge from the current impulse in one exemplary implementation. It is observed that the CPE voltage trace 1062 takes the form of a voltage transient with a magnitude that decays over time. Differences in the impedances of CPE1 and CPE2 produce non-matching voltage transients (i.e., Vᵢₙ₁−Vᵢₙ₂≠0), and, in response to receiving the non-matching transient signals at each input 1001a and 1001b, the measurement amplifier 1011 produces an artefact in the output voltage (referred to as “mismatch artefact”). The presence of mismatch artefact may prevent the measurement of an evoked neural response, or otherwise obscure or distort the value of any measured parameter of the neural response. It is therefore desired to eliminate or reduce mismatch artefact occurring at the measurement amplifier.

Various approaches have been previously implemented to address the problem of mismatch artefact, including limiting or preventing its occurrence or mitigating its effect at the inputs to a differential ECAP measurement amplifier. In one approach, as presented in International Patent Publication No. WO2014/071445, mismatch artefact is lowered by decreasing the amount of charge injected on the measurement electrodes during stimulation. By maintaining the common mode potential on the tissue at a constant voltage, this “virtual ground” approach mitigates potential difference variation across the CPEs to prevent the occurrence of voltage transients.

In another approach, as presented in International Patent Publication No. WO2015/168735, the input impedances at the measurement amplifier are set sufficiently high such as to mitigate artefact in relation to the expected neural responses. That is, a minimum value for the input impedance Zᵢₙ is determined based on the impedance of the CPE of the measurement electrode, the expected magnitude of the neural response sensed by the measurement electrode, and the differential voltage at the electrode-tissue interfaces due to the stimulation.

In a further approach, as presented in International Patent Publication No. WO2022/217322, the difference in impedance values of the CPEs is compensated by adding an impedance to respective inputs of the measurement amplifier. That is, artefact is reduced to zero, or close to zero, by separately adjusting the input impedances Zᵢₙ₁ and Zᵢₙ₂, using added resistive and capacitive elements to match a ratio of CPE impedance to input impedance values across each amplifier input path, thereby compensating for mismatch in the CPE impedances that would otherwise generate mismatch artefact. It is noted that in this approach, and all other similar prior approaches, the impedances added to the amplifier inputs are positive (real or complex) values. It is desired to develop an approach for measuring evoked neural responses that improves on the prior art in relation to addressing mismatch artefact, or that at least provides a useful alternative.

### Overview of the Disclosed Technology

Disclosed herein are methods, devices, and systems for measuring an evoked neural response by providing a negative impedance to one or more inputs of an amplifier (referred to as a “measurement amplifier”) configured to amplify a voltage associated with the neural response. In some examples described herein, the measurement amplifier has two inputs, each of which is exposed to a voltage transient resulting from a current induced at a measurement electrode-tissue interface by variation in a common mode potential of the tissue (differential configuration). In other examples, only one input of the measurement amplifier is exposed to a voltage transient, for example as a result of the other input being connected to an indifferent electrode, that is, an electrode not subject to, or not susceptible to, common mode tissue voltage fluctuations induced by the stimulation (crosstalk). By adding a negative impedance in parallel with the existing input impedance at each transient-exposed input of the measurement amplifier according to the techniques described herein, the flow of undesired currents through the respective electrode-tissue interfaces is reduced or prevented, thereby decreasing or eliminating mismatch artefact in measurement of evoked neural responses.

Measurement of an evoked neural response may be performed by an implantable device, or by a neural stimulation system using an implantable device. The device includes a stimulus source configured to deliver neural stimuli via one or more stimulus electrodes to neural tissue, the neural stimuli being configured to evoke a neural response from the neural tissue. A measurement amplifier of the device is configured to amplify a signal sensed between a first input of the measurement amplifier (via a first measurement electrode) and a second input of the measurement amplifier (via a second measurement electrode) subsequent to a provided neural stimulus, where the sensed signal comprises the evoked neural response. One or more impedance elements are configured to provide a negative impedance to at least one of the first and second inputs of the measurement amplifier. The stimulus source is controlled by a control unit of the device, the control unit configured to deliver a neural stimulus, and to measure the evoked neural response of the amplified sensed signal.

In some examples, the impedance elements consist of a first set of impedance elements {Zₐ₁₁, . . . , Zₐ_{₁ₙ}} and a second set of impedance elements {Zₐ₂₁, . . . , Zₐ₂ₘ} that are independently connected to corresponding first and second inputs of the measurement amplifier. For example, each set of impedance elements may be connected in parallel with an input impedance Zᵢₙ of a measurement amplifier input, and between the measurement amplifier input and a corresponding measurement electrode. This enables equivalent first and second negative impedances Zₐ₁ and Zₐ₂ to be provided to the respective first and second inputs of the measurement amplifier. In other examples, a single set of one or more impedance elements is configured to provide a single negative impedance Zₐ to a single input of the measurement amplifier.

The negative impedance Zₐ at a given input of the measurement amplifier is provided as an added impedance relative to the input impedance (i.e., an equivalent circuit impedance at the input). Further, the value(s) of the negative impedance(s) provided by the one or more impedance elements are configurable, and may be set to increase the total impedance at the respective measurement amplifier input. In some examples, the added negative impedance results in a total impedance at each respective input that is at least equal to a threshold impedance value. The threshold impedance value may be predetermined based on the characteristics of the neuromodulation or associated devices or systems, to suppress a transient voltage generated at an electrode-tissue interface of the measurement electrode as a result of the neural stimulus to a degree that enables measurement of the evoked neural response.

In another example, the value of the added impedance Zₐ is equal, or substantially equal, to the negative of the value of the existing input impedance Zᵢₙ at a given measurement amplifier input (i.e., Zₐ=−Zᵢₙ), such that the total impedance Zₜₒₜₐₗ is driven towards an infinite value. This is advantageous in that a voltage transient generated at the electrode-tissue interface of the corresponding measurement electrode is eliminated or at least substantially reduced.

The addition of negative impedances to the measurement amplifier inputs advantageously enables improved measurement of evoked neural responses by addressing mismatch artefact without: explicitly controlling for undesired variation in the common mode potential; requiring knowledge of expected neural response voltages or mismatch differential voltages; or compensating for any relative difference in the impedance of the measurement electrodes (i.e., the CPE impedances) which may change unexpectedly.

The negative impedances are potentially complex impedances that may be generated virtually based on Miller's theorem, for example by implementing one or more negative impedance generator circuits. In some configurations, each negative impedance generator circuit is coupled to an input of the measurement amplifier. In some examples, a negative impedance generator circuit includes a Miller amplifier having a Miller impedance element Zₘ connected across its input and its output. A gain of the Miller amplifier is such that the effective input impedance to ground at the input of the Miller amplifier provides the negative impedance Zₐ to the measurement amplifier input in response to a connection of the impedance generator circuit to the measurement amplifier input. The use of a Miller amplifier advantageously results in a low power consumption particularly in applications where the input impedance of the measurement amplifier is substantially capacitive.

Further, the value of a negative impedance Zₐ provided by a Miller-based negative impedance generator circuit is adjustable by adjusting the gain of the Miller amplifier. For example, when the input impedance of the measurement amplifier is known to be Zᵢₙ, the added impedance Zₐ may be adjusted to be equal to, or to approach, −Zᵢₙ driving the total impedance Zₜₒₜₐₗ at the input towards an infinite value. However, the value of Zᵢₙ may be unknown or may change over time from an initially known value (e.g., due to device-to-device variability). In such or other situations, the value of the added impedance Zₐ provided by the impedance generator circuit may be adjusted via adjusting a gain value of the Miller amplifier. This advantageously enables dynamic modification of the impedance realised at the measurement amplifier input thereby improving the ability to suppress or eliminate the effect of voltage transients on neural response measurement.

Various configurations of one or more impedance generator circuits may be used to provide added negative impedance to inputs of an amplifier configured to measure evoked neural responses, according to the examples presented herein. In some examples, first and second impedance generator circuits are independently connected to the respective first and second inputs of the measurement amplifier. This enables the total impedance at each measurement amplifier input to be independently controlled (e.g., by adjusting the effective impedance value provided by the connected impedance generator circuit).

In other examples, the first and the second input of the measurement amplifier are both connected to a common Miller amplifier. In some examples, the common Miller amplifier may drive a plurality of individual impedance elements, each providing a separate Miller impedance to the common Miller amplifier. In some examples, the impedance elements may be arranged in a star configuration across the first input and the second input of the measurement amplifier. In some examples, the impedance elements may comprise one or more filter capacitors each having a predetermined capacitance. The use of a common Miller amplifier to provide negative impedance to both measurement amplifier inputs is advantageous in reducing the circuitry that is required to achieve improved neural response measurement (i.e., by eliminating or reducing mismatch artefact). This also enables neuromodulation devices and systems, such as for example an implantable neurostimulation device, to be constructed with a reduced size and/or with a lower power consumption compared to the use of independent first and second impedance generator circuits.

Neuromodulation Device with Added Negative Impedance

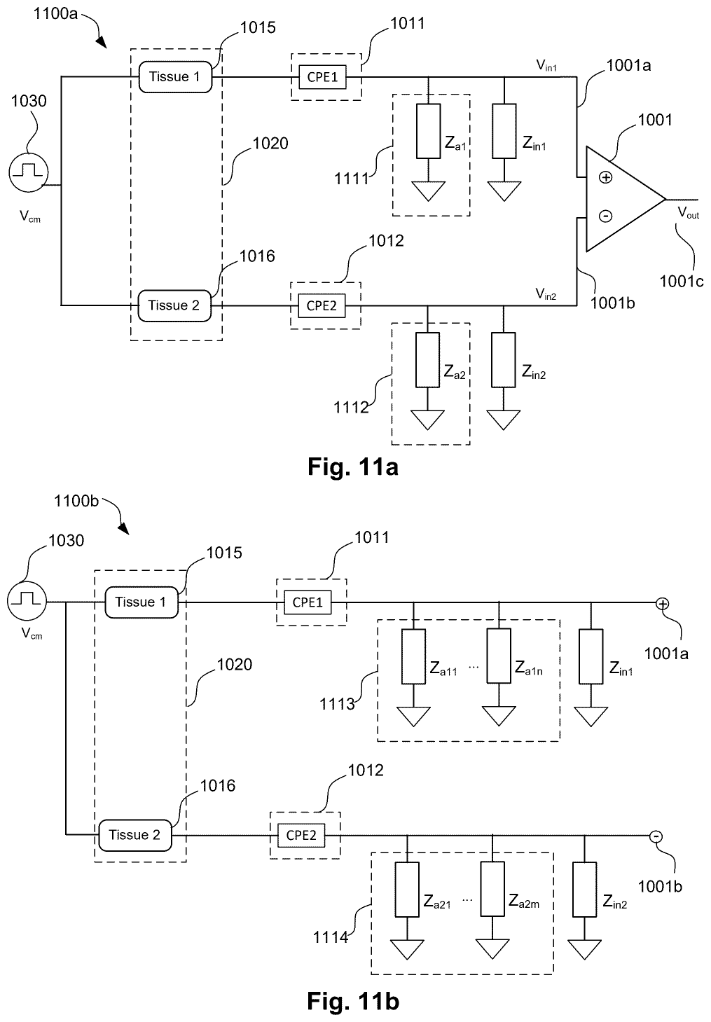

FIG. 11a illustrates electrical circuitry 1100a for measuring an evoked neural response according to the proposed techniques. One or more stimulus electrodes provide a common mode potential 1030 of magnitude V_{cm} to neural tissue 1020, where the neural tissue comprises first and second tissue components 1015 and 1016. First and second measurement electrodes 1011 and 1012 are in contact with first and second tissue components 1015 and 1016 at respective first and second electrode-tissue interfaces represented by CPE1 and CPE2. Measurement amplifier 1001 has first and second inputs 1001a and 1001b connected to first measurement electrode 1011 and second measurement electrode 1012 respectively, and an output 1001c.

Input voltages Vᵢₙ₁ and Vᵢₙ₂ sensed at first and second measurement amplifier inputs 1001a and 1001b produce an output voltage Vₒᵤₜ proportional to the voltage differential as

$${V}_{out} = {A}_{m} ( {V}_{in 1} - {V}_{in 2} ) \qquad ( 5 )$$

where Aₘ is the measurement amplifier gain. First and second measurement amplifier inputs 1001a and 1001b have corresponding existing input impedances Zᵢₙ₁ and Zᵢₙ₂. As measurement amplifier 1001 is non-ideal, existing input impedances Zᵢₙ₁ and Zᵢₙ₂ are finite.

The total input impedance of measurement amplifier 1001 is increased by adding one or more impedance elements to provide a negative impedance Zₐ in parallel to each existing input impedance Zᵢₙ₁ and Zᵢₙ₂ of the measurement amplifier 1001. Various configurations of the one or more impedance elements may be realised to produce equivalent negative impedances 1111 and 1112 as shown in FIG. 11a. In the example electrical circuitry 1100a shown in FIG. 11a, first and second negative impedances 1111 and 1112 of values Zₐ₁ and Zₐ₂ respectively are added to the first and second measurement amplifier inputs 1001a and 1001b respectively.

FIG. 11b illustrates an example circuit model in which a first set 1113 of n impedance elements {Zₐ₁₁, . . . , Zₐ_{₁ₙ}} are connected to the first measurement amplifier input 1001a and a second set 1114 of m impedance elements {Zₐ₂₁, . . . , Zₐ₂ₘ} are connected to the second measurement amplifier input 1001b. Individual impedances within the first and second sets 1113 and 1114 may be complex impedances, and may have any combination of parallel or series connections. For example, in response to a parallel arrangement of the individual intra-set impedance elements, as depicted in FIG. 11b, the effective negative impedances Zₐ₁ and Zₐ₂ are given by:

$${Z}_{a 1} = \frac{1}{{\sum }_{i = 1}^{n} \frac{1}{{z}_{a 1 i}}} \qquad ( 6 )$$

$${Z}_{a 2} = \frac{1}{{\sum }_{j = 1}^{m} \frac{1}{{z}_{a 2 j}}} \qquad ( 7 )$$

In some examples, the negative impedances Zₐ₁ and Zₐ₂ have the same values (i.e., Zₐ₁=Zₐ₂). Alternatively, the values of the negative impedances Zₐ₁ and Zₐ₂ may differ (i.e., Zₐ₁≠Zₐ₂), for example depending on the input impedance Zᵢₙ₁ and Zᵢₙ₂ at each input 1001a and 1001b of measurement amplifier 1001.

The addition of a negative impedance effectively increases the input impedance at each input of the measurement amplifier 1001. At a given input of the measurement amplifier 1001, the total input impedance can be calculated as:

$${Z}_{total} = \frac{{Z}_{a} {Z}_{in}}{{Z}_{a} + {Z}_{in}} \qquad ( 8 )$$

As Zₐ approaches −Zᵢₙ from −∞, Zₜₒₜₐₗ will increase from Zᵢₙ (i.e., a value of Zₐ within this range will always cause Zₜₒₜₐₗ to be greater than Zᵢₙ) towards infinity. If Zₐ=−Zᵢₙ, then the total input impedance is infinite:

$${Z}_{total} = \frac{- {Z}_{in} {Z}_{in}}{{Z}_{in} + {Z}_{in}} = \frac{- {Z}_{in}^{2}}{0} = ∞ \qquad ( 9 )$$

Therefore, by setting the value of the added impedance Zₐ to approach, or to be equal to, the negative of the value of the existing input impedance Zᵢₙ, the total impedance Zₜₒₜₐₗ at the given measurement amplifier input is driven towards an infinite value.

### Negative Impedance Generator Circuit

In some implementations of devices and systems for measuring evoked neural responses according to the proposed techniques, the impedances provided to the first and second measurement amplifier inputs are generated by electronic circuitry referred to herein as an “impedance generator circuit”. A negative impedance generator circuit is an impedance generator circuit configured to use Miller's theorem to provide a virtual negative impedance value across its respective inputs.

Miller's theorem shows that an amplifier with an impedance connected between its input and output can equivalently be represented by an amplifier with two impedances, one from each of input and output to some reference node.

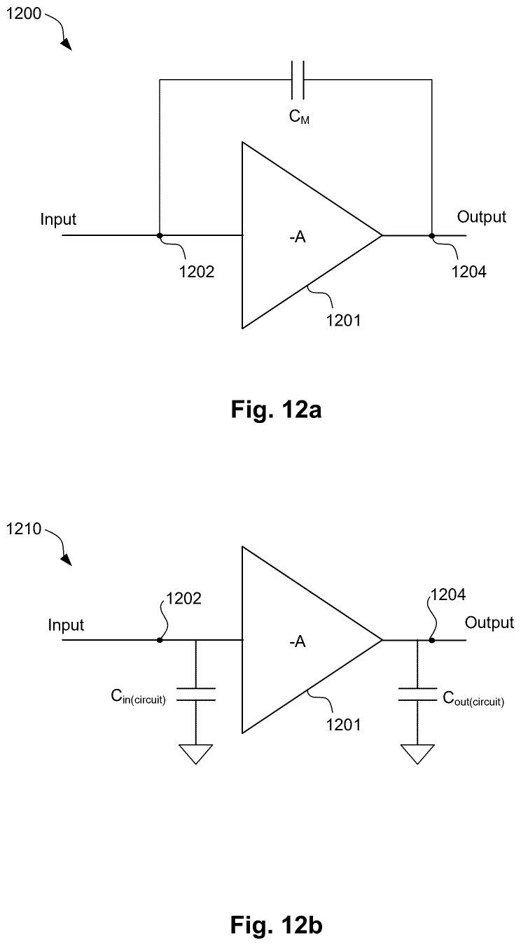

FIG. 12a illustrates an impedance generator circuit 1200 comprising a Miller amplifier 1201 with gain −A and having an input terminal 1202 and output terminal 1204 with a Miller impedance Cₘ connected between the input 1202 and the output 1204. It will be appreciated that, although the Miller impedance is depicted as a capacitive element in FIG. 12a, generally the Miller impedance may be a complex impedance Zₘ containing resistive and reactive (inductive or capacitive) components.

FIG. 12b illustrates an impedance generator circuit 1210 that according to Miller's Theorem is electrically equivalent to the impedance generator circuit 1200 of FIG. 12a, where the equivalent impedances at input 1202 and output 1204 (Z_{in(circuit)} and Z_{out(circuit)} respectively) are a function of the gain A and the Miller impedance Zₘ. For a capacitive Miller impedance Cₘ as depicted, the equivalent impedances are given by:

$${C}_{in ( circuit )} = {C}_{M} ( 1 + \vert A \vert ) \qquad ( 10 )$$

$${C}_{out ( circuit )} = {C}_{M} ( 1 + 1 / \vert A \vert ) \qquad ( 11 )$$

Miller's theorem can be used to describe several circuit phenomena, including the Miller effect and negative impedance generation.

Let K=−A, where K is the non-inverting gain of the Miller amplifier 1201. Then

$${Z}_{in ( circuit )} = \frac{{Z}_{M}}{1 - K} \qquad ( 12 )$$

$$and$$

$${Z}_{out ( circuit )} = \frac{{Z}_{M}}{1 - \frac{1}{K}} \qquad ( 13 )$$

If A<−1 (i.e., K>1) then Z_{in(circuit)}<0 (i.e. the equivalent input impedance of the Miller amplifier 1201 is negative). If −1<A<0 (i.e., 0<K<1) then Z_{out(circuit)}<0 (i.e. the equivalent output impedance of the Miller amplifier 1201 is negative).

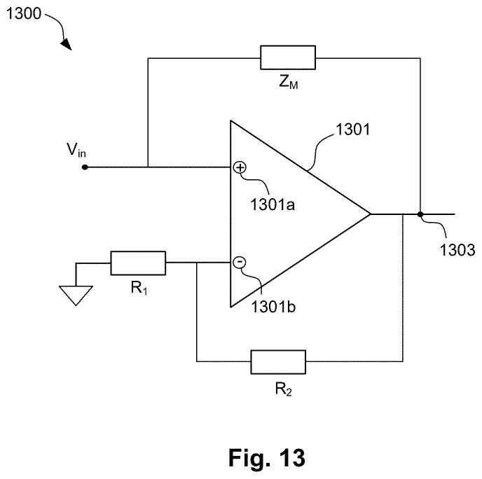

FIG. 13 illustrates an exemplary negative impedance generator circuit 1300 (also referred to as a “negative Z-generator”). Differential amplifier 1301 (referred to herein as an “op-amp”) has a first (non-inverting) input 1301a and a second (inverting) input 1301b. Miller impedance Zₘ is connected across non-inverting input 1301a and an output 1303. A pair of resistors R₁ and R₂ are connected in series between ground and the output 1303, having an intermediate connection with inverting input 1301b.

The Miller amplifier of the negative Z-generator circuit 1300 has a (positive) gain K with a value based on the value of the resistors R₁ and R₂:

$$K = 1 + {R}_{2} / {R}_{1} \qquad ( 14 )$$

As R₁ and R₂ are real positive values, gain K is always greater than 1. The effective input impedance to ground at the non-inverting input 1301a of the negative impedance generator circuit 1300 depicted in FIG. 13 is:

$${Z}_{in ( circuit )} = - {Z}_{m} \frac{{R}_{1}}{{R}_{2}} \qquad ( 15 )$$

When R₁=R₂, the gain of the Miller amplifier is 2 and Z_{in(circuit)}=−Zₘ. Miller impedance Zₘ can be a resistor, capacitor, inductor, or a network of impedances. The value of Z_{in(circuit)} is dependent on the Miller impedance Zₘ and the gain K of the Miller amplifier. For a given value of Zₘ, the value of Z_{in(circuit)} approaches negative infinity as the gain K decreases towards 1 (i.e., for R₂<<R₁).

### Independent Negative Impedance Generators

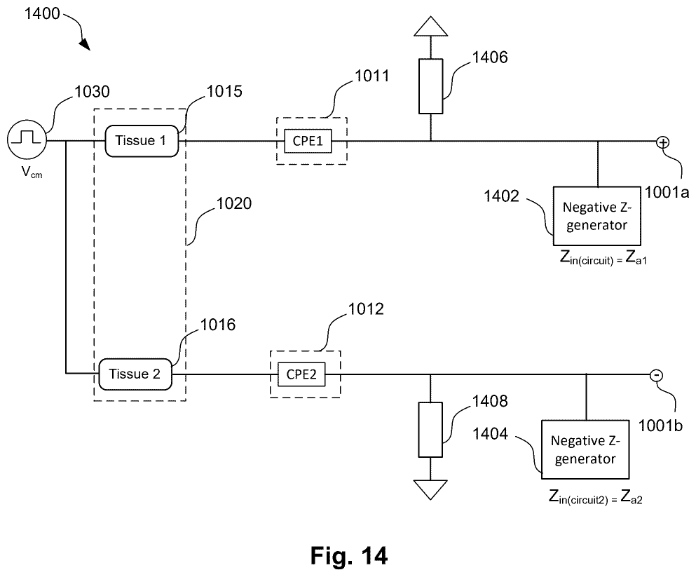

FIG. 14 illustrates a circuit model 1400 for measurement of neural responses similar to the models 1100a and 1100b of FIGS. 11a and 11b and with a plurality of independent negative impedance generator circuits connected to the respective inputs 1001a and 1001b of measurement amplifier (not shown in FIG. 14).

In the example circuit model 1400 of FIG. 14, the first input 1001a of the measurement amplifier is connected to a first negative impedance generator circuit (negative Z-generator) 1402 and the second input 1001b of the measurement amplifier 1001 is connected to a second negative impedance generator circuit 1404. The negative Z-generators 1402 and 1404 are added in parallel with existing impedance elements 1406 and 1408 representing the input impedances Zᵢₙ₁ and Zᵢₙ₂ respectively of the measurement amplifier. Each negative Z-generator 1402 and 1404 provides an effective input impedance Z_{in(circuit)} at the respective inputs 1001a and 1001b of measurement amplifier that corresponds to the negative impedances Zₐ₁ and Zₐ₂ depicted in FIG. 11a.

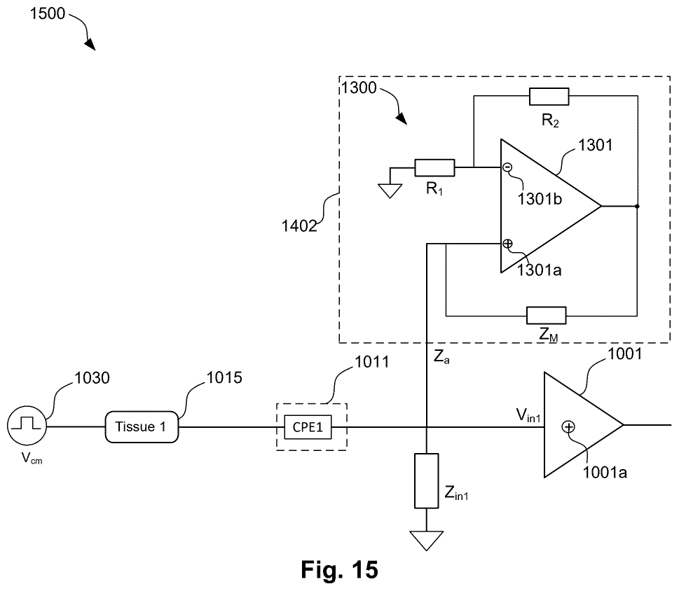

FIG. 15 illustrates a circuit model 1500 that is equivalent to one half of the model 1400 of FIG. 14, showing the addition of the negative Z-generator 1402, implemented as circuit 1300, to the positive input 1001a of the measurement amplifier 1001. The same circuit 1300 is added, as the negative Z-generator 1404, to the negative input 1001b of the measurement amplifier 1001, however this is omitted from display in FIG. 15 for simplicity. Setting R₁=R₂, and the Miller impedances Zₘ to Zᵢₙ₁ and Zᵢₙ₂ respectively, the gain of the Miller amplifier of the circuit 1300 is K=2 and the input impedances provided by the negative Z-generators 1402 and 1404 are −Zᵢₙ₁ and −Zᵢₙ₂ respectively. Consequently, the total input impedance Zₜₒₜₐₗ at the measurement amplifier inputs 1001a and 1001b will be driven to infinity.

Alternatively, the circuit model 1500 in FIG. 15 may be taken to show the addition of the negative Z-generator 1402, implemented as circuit 1300, to the positive input 1001a of the measurement amplifier 1001, while the negative input 1001b is connected to an electrode not exposed to a voltage transient (not shown).

In some examples, the Miller impedance Zₘ is substantially capacitive (represented by a capacitor Cₘ) equal to the total input capacitive impedance to ground (Cᵢₙ) at the input of the measurement amplifier 1001 to which it is connected.

### Simulating the Impedance Generator Circuit

Simulation of the circuit model 1500 was performed in the SPICE package, using a voltage-controlled voltage source (VCVS) (ideal amplifier) with a gain of 100 k as the amplifier 1301. A second simulation was performed with a single pole at 100 Hz added to the amplifier 1301 to form a realistic op-amp. A single pole at 100 Hz gives the Miller amplifier of FIG. 13 a GBW of 10 MHz. For the purposes of comparison, a simulation was performed of the model 1500 without negative impedance provided to the measurement amplifier inputs (i.e., representing the configuration 1000 of FIG. 10b). The circuit parameters used are listed in Table 1.

TABLE 1
SPICE Parameters for simulating the neural response measurement
circuit models of FIG. 10b (without negative impedance)
and of FIG. 15 (with negative impedance).

| Parameter | Value | |
|---|---|---|
| V_{cm} | 15 V Pulse, 200 μs Width, 100 μs Delay, 1 μs Rise/Fall Time | |
| Zₜᵢₛₛᵤₑ | 250Ω | |
| α (CPE) | 0.636 | |
| C_{f} (CPE) | 30 | μF/s^{1-α} |
| Zᵢₙ | 100 | pF |
| R₁, R₂ | 1 | kΩ |

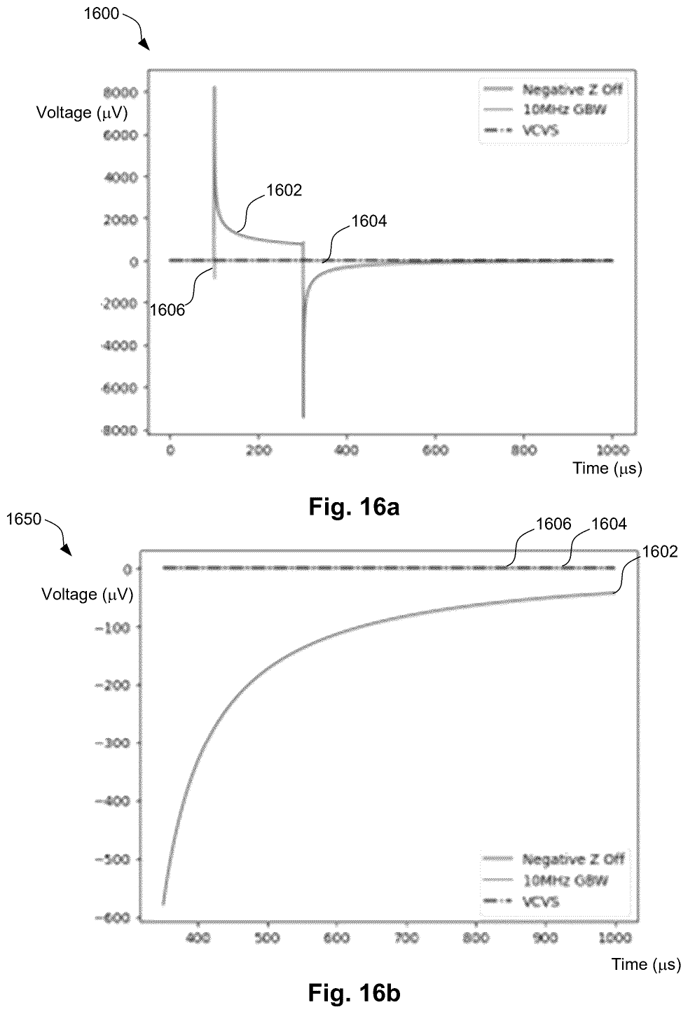

FIG. 16a is a graph 1600 containing plots 1602, 1604, 1606 of voltage across the CPE 1011 representing a corresponding measurement electrode during a 1 ms simulation of the models of FIG. 10b and FIG. 15. The plot 1602 shows the CPE voltage without negative impedance added to the measurement amplifier input (i.e., the model of FIG. 10b). The plots 1604 and 1606 show the CPE voltage with negative impedance added to the measurement amplifier input (i.e., the model of FIG. 15) using either the VCVS (ideal amplifier) or realistic op-amp implementations respectively. FIG. 16b is a graph 1650 illustrating the voltage plots of FIG. 16a commencing 50 μs after the end of the V_{cm} pulse. A clear transient is observed in the voltage plot 1602 associated with the step change in V_{CM}, in contrast to plots 1606 and 1604 which show the effect of the added negative impedance in reducing or eliminating the voltage transient respectively.

Comparing the plots 1602 and 1606 of FIGS. 16a and 16b suggests that the mismatch artefact will be reduced when a negative impedance is added at each measurement amplifier input of a differential configuration. With the negative impedance disconnected, the peak-to-peak voltage 50 μs after the V_{cm} pulse was recorded as approximately 600 uV. In contrast, the peak-to-peak voltage was virtually zero when either the VCVS or realistic op-amp implementation of the negative Z-generator circuit was connected to the measurement amplifier input. In response to use of a negative Z-generator according to the VCVS implementation, the charge injected into the CPE was negligible for the duration of the simulation. In response to adding a 100 Hz pole to the VCVS implementation of the negative Z-generator, such that the GBW (speed) of the Miller amplifier is 10 MHz, a small impulse is observed on the CPE voltage transient at each pulse edge. Since this elimination or reduction of the voltage transient across the CPE is insensitive to the actual parameters of the CPE, it may be inferred that the difference between the two CPE voltage transients in a differential measurement configuration such as the circuit 1000 illustrated in FIG. 10b with some mismatch between the two CPEs (the mismatch artefact) would also be reduced or eliminated.

### Effect of the Amplifier Speed

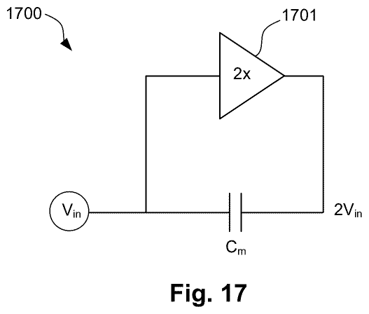

FIG. 17 illustrates a circuit model of an impedance generator circuit 1700 where the resistance values of resistors R₁ and R₂ (not shown) are set to be equal. Circuit 1700 has internal voltages comprising an input voltage Vᵢₙ and an output voltage Vₒᵤₜ=2Vᵢₙ at the respective terminals of the Miller amplifier 1701 which has a gain of K=2. The charge stored on the Miller capacitor Cₘ of the impedance generator circuit 1700, is defined by the voltage across its terminals:

$$Q = C V = {C}_{m} ( {V}_{in} - 2 {V}_{in} ) = - {C}_{m} {V}_{in} \qquad ( 16 )$$

The charge stored is a function of Cₘ, Vᵢₙ and the gain of the Miller amplifier 1701. In response to a step change in Vᵢₙ, the speed of the Miller amplifier 1701 affects how quickly the programmed gain is produced but not the gain itself.

In some examples, the speed of the Miller amplifier 1701 of the negative impedance generator circuit 1700 is set to be sufficiently fast to prevent charging of CPE1 (or CPE2) in response to the current impulses from the common mode potential V_{CM}. However, it is a characteristic of the proposed techniques that even if a charge is applied to the CPE from a change in the common mode potential V_{CM}, if the charge is reversed before it has spread through the CPE then artefact at the measurement amplifier input is avoided.

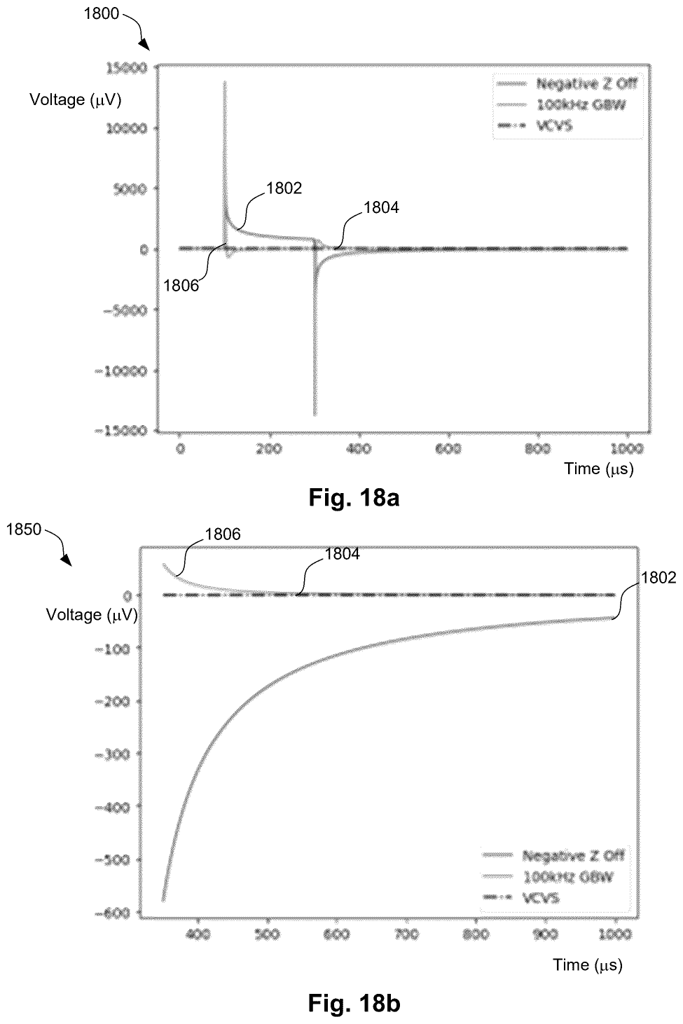

To demonstrate this behaviour, simulation was performed with the pole of the op-amp shifted from 100 Hz to 1 Hz (i.e., the Miller amplifier 1701 was effectively slowed by two orders of magnitude from a GBW of 10 MHz to a GBW of 100 kHz). FIG. 18a is a graph 1800 containing plots 1802, 1804, 1806 of voltage across a CPE connected to a corresponding measurement electrode during a 1 ms simulation, where the pole of the op-amp was set to 1 Hz. FIG. 18b illustrates the voltage plots of FIG. 18a commencing 350 μs after the end of the V_{cm} pulse.

In the simulation, the Miller amplifier is sufficiently slow that it increases the load on the CPE during the V_{cm} step. The CPE transient with the GBW of 100 kHz (plot 1806) is almost double in peak size (14000 μV vs 8000 μV) compared to when the GBW was 10 MHz (plot 1606 in FIG. 16a). Despite this, as shown in FIGS. 18a and 18b, there is still a significant reduction in the post-pulse artefact (i.e., by a factor of almost 10) compared to when the negative impedance is disconnected. That is, despite being slowed by the placement of the pole at 1 Hz, the Miller amplifier slowly reverses the charge after each impulse.

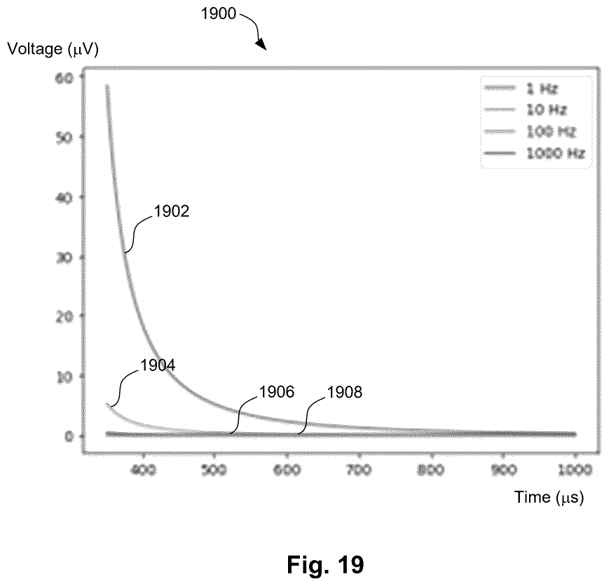

FIG. 19 is a graph 1900 containing plots 1902, 1904, 1906 of CPE voltage as the pole frequency of the op-amp was swept from 1 Hz to 1 kHz (and hence the GBW of the Miller amplifier from 100 kHz to 100 MHz) in a simulation. Plots 1902, 1904, 1906 and 1908 show the CPE voltage for a pole frequency of the op-amp at 1 Hz, 10 Hz, 100 Hz and 1000 Hz respectively. The voltages are shown 50 μs after the end of the V_{cm} pulse. For a pole frequency above 100 Hz, the artefact is negligible. For a pole frequency at 10 Hz, the artefact is around 100 times smaller (around 6 μV vs 600 μV) than when no negative impedance is provided to the corresponding measurement amplifier input.

The simulation results depicted in FIGS. 18a, 18b and 19 show that, provided that the variation in V_{CM} is not too rapid, then the presence of a negative impedance generator will reverse the charge of the impulse generated by the change in V_{CM}, and therefore result in reduction or elimination of mismatch artefact, even if the Miller amplifier 1701 is too slow to prevent CPE charging.

### Effect of the Amplifier Gain

Referring to the impedance generator circuit models depicted in FIGS. 13 and 17, in some examples the Miller impedance element Zₘ (depicted in FIG. 17 as a capacitive element Cₘ) is set to produce a negative input impedance Z_{in(circuit)} that is equal to the measurement amplifier input impedance Zᵢₙ. In other examples, the Miller impedance (capacitance Cₘ) does not need to equal the measurement amplifier input impedance Zᵢₙ. The gain K of the Miller amplifier 1701 can be used to tune the negative impedance produced by the circuit 1300 or 1700. For example, the input capacitance of the negative impedance generator can be calculated with:

$${C}_{in ( circuit )} = {C}_{m} ( 1 - K ) \qquad ( 17 )$$

where Cₘ is the Miller capacitance, C_{in(circuit)} is the effective input impedance of the circuit (as provided to the connected measurement amplifier input) and K is the (non-inverting) gain of the Miller amplifier. Equation (17) can be rearranged for gain:

$$K = 1 - \frac{{C}_{in ( circuit )}}{{C}_{m}} \qquad ( 18 )$$

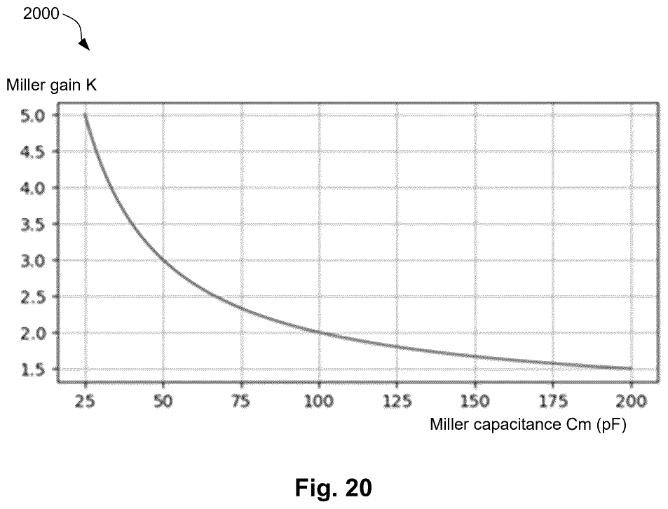

FIG. 20 is a graph 2000 of the Miller gain K required for a given Miller capacitance Cₘ to generate a negative input capacitance C_{in(circuit)} of −100 pF. If Cₘ>>|Cᵢₙ|, where Cᵢₙ is the equivalent capacitance at the measurement amplifier input to which C_{in(circuit)} is matched, then the gain required approaches 1. As Cₘ approaches zero, the Miller gain K required approaches infinity.

With reference to FIG. 13, the negative impedance value produced by the negative Z-generator circuit 1300 may therefore be controlled by using one or more techniques to adjust the gain K of the Miller amplifier. In some examples, resistors R₁ and R₂ are resistors with resistance values that are selectable over a predetermined range (e.g., 0.1-10 kΩ). In some examples, the values of the resistors R₁ and R₂ are adjustable or configurable from an initially selected or manufactured value. Selecting or adjusting the values of R₁ and R₂ within the predetermined range of resistances in turn selects or adjusts the gain K, thereby enabling the effective input impedance Z_{in(circuit)} of the circuit 1300 to be tuned to a desired value (e.g., in real-time during neural stimulation).

In some examples, the gain of the Miller amplifier is adjusted to set the effective input impedance Z_{in(circuit)} of the circuit 1300 such that the total impedance Zₜₒₜₐₗ at the input of the measurement amplifier is at least a threshold impedance value Z_{T}. The threshold impedance value may be determined as the impedance value that ensures that the voltage arising across the CPE in response to the change in common mode potential is constrained to a level which permits assessment of the neural response voltage seen at the corresponding measurement electrode. For example, a value for the threshold impedance may be determined by following the approach described in International Patent Publication No. WO2015/168735 as:

$${Z}_{T} = \frac{{Z}_{c} ( {V}_{s 1} - {V}_{s 2} )}{{V}_{E}} \qquad ( 19 )$$

where Z_{c} is the impedance of the CPE of each measurement electrode, Vₛ₁−Vₛ₂ is the differential voltage across CPE1 and CPE2 as a result of the stimulus, and V_{E} is the differential neural response voltage seen at the measurement electrodes. This enables mitigation or suppression of voltage transients at the corresponding measurement electrodes without requiring Zₜₒₜₐₗ to be driven towards an infinite value, given knowledge of the characteristics of the measurement electrodes and the expected differential neural response and mismatch artefact voltages.

### Effect of the Magnitude of the Common Mode Potential

In the example simulations of FIGS. 16a, 16b, 18a, 18b, and 19, V_{cm} has a maximum magnitude of the system supply voltage of VDDHV (e.g., 15V). The impedance generator circuit 1300 depicted in FIG. 13 must therefore produce a voltage that is greater than the VDDHV value as long as the Miller amplifier gain K is greater than unity, which it must be to generate negative impedance. This means the Miller amplifier needs to be able to produce voltages greater then VDDHV without saturating. This in turn would require the Miller amplifier to be powered off a supply rail with a voltage greater than VDDHV.

In some implementations of a stimulator 100, V_{cm} will have a magnitude lower than VDDHV, thereby advantageously allowing the negative impedance generator circuit 1300 to be powered off the existing VDDHV rail of the stimulator 100 without saturating the Miller amplifier.

### Common Miller Amplifier

Considering FIG. 15, and assuming that the impedances of CPE1 and CPE2 are negligible compared to either the input impedances Zᵢₙ₁ and Zᵢₙ₂ or the Miller impedance Zₘ of circuit 1300, the input of the Miller amplifier of circuit 1300 may be connected to V_{cm} or to an input of the measurement amplifier 1001 (as in FIG. 15) with equivalent effect.

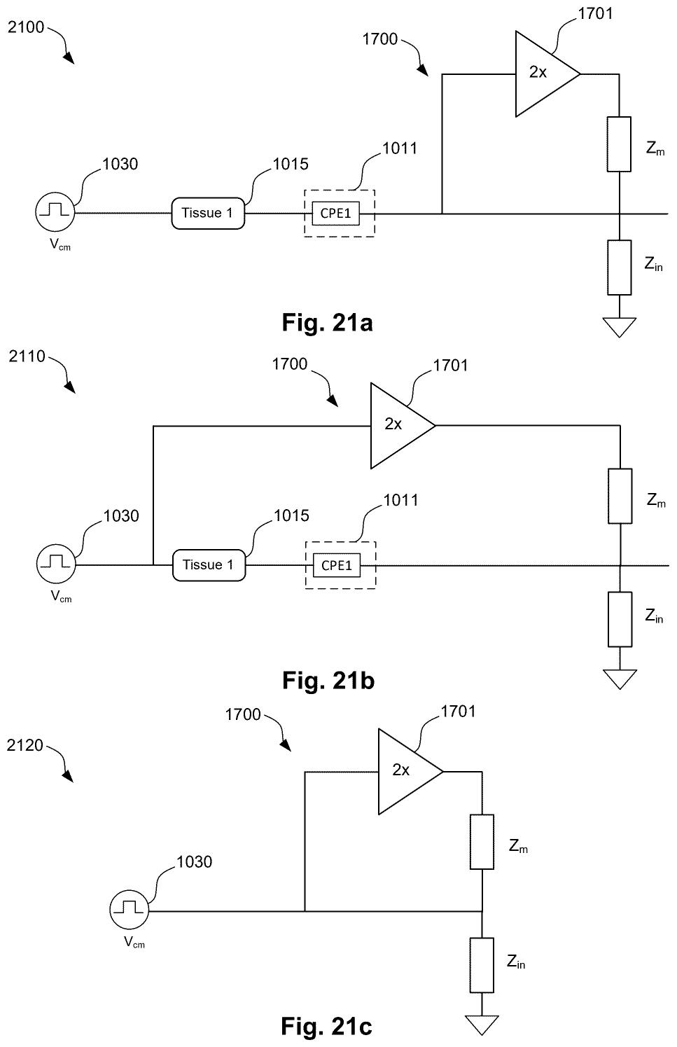

FIGS. 21a and 21b illustrate models 2100 and 2110 of a negative Z-generator circuit 1700 connected to the measurement amplifier input and V_{cm} respectively. FIG. 21c illustrates a model 2120 containing a negative Z-generator circuit 1700 that is approximately electrically equivalent to the circuits of FIGS. 21a and 21b under the given assumptions.

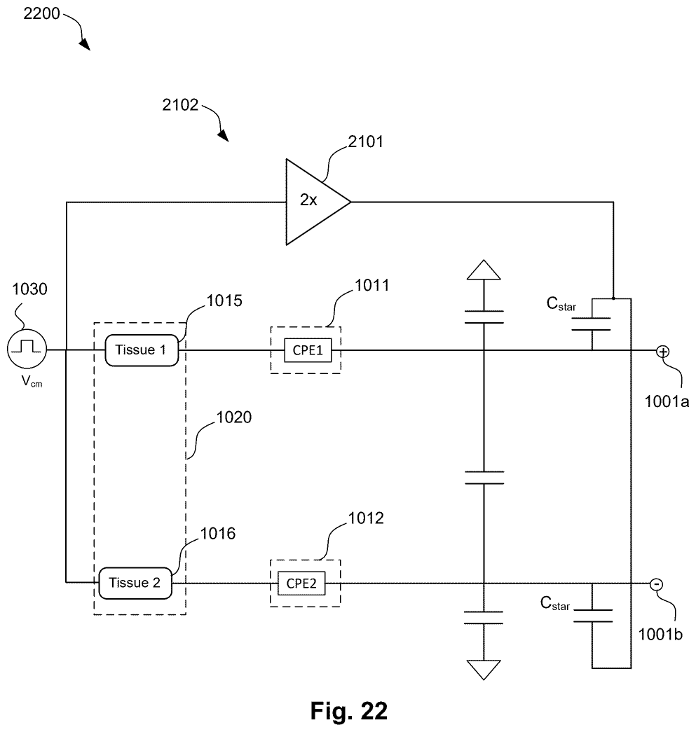

FIG. 22 illustrates a circuit model 2200 in which a common Miller amplifier 2101 drives a star point of a plurality of impedances, which are implemented as a network of capacitors Cₛₜₐᵣ in the circuit model 2200 depicted by FIG. 22, arranged in a star configuration across the inputs 1001a and 1001b of the measurement amplifier 1001 (also referred to as a “star point driver” implementation of the present technology). In some examples, the network of capacitors comprises one or more filter capacitors, for example each with a capacitance value of at least 100 pF. The star capacitor network provides a plurality of impedances with an equivalent impedance forming the Miller impedance element of the negative Z-generator circuit 2102. In the example circuit model 2200 of FIG. 22, in contrast to the model 1500 of FIG. 15, the input of the common Miller amplifier 2101 is connected to the common mode potential source 1030 rather than the respective inputs 1001a and 1001b of the measurement amplifier 1001. In an exemplary implantable device, the common mode potential source 1030 may be accessed through a case electrode or an unused stimulation electrode.

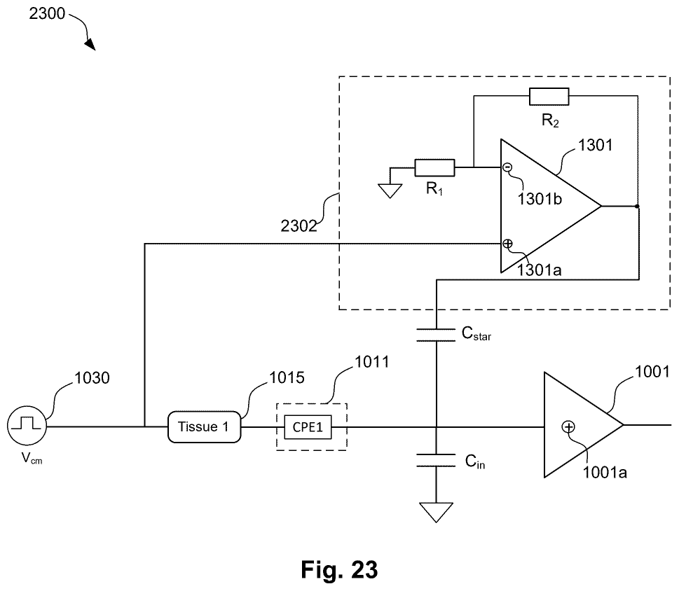

FIG. 23 illustrates a circuit model 2300 equivalent to the model 2200 of FIG. 22, showing the connection of the common Miller amplifier 2302 via the capacitive element Cₛₜₐᵣ to the positive input 1001a of the measurement amplifier 1001, thereby providing a negative Z-generator to the positive input 1001a. There is no additional circuitry required to provide a negative impedance to the negative input 1001b (not shown) because the common Miller amplifier 2302 is connected to both inputs 1001a and 1001b via the respective capacitive elements Cₛₜₐᵣ (i.e., providing the same added negative impedance to each). For example, by setting R₁=R₂, the gain of the common Miller amplifier is K=2 and the equivalent input impedance (resulting from setting each capacitive element Cₛₜₐᵣ equal to Cᵢₙ) provided by the negative Z-generator 2102 is −Zᵢₙ. Consequently, the total input impedance Zₜₒₜₐₗ at the measurement amplifier inputs 1001a and 1001b will be driven to infinity.

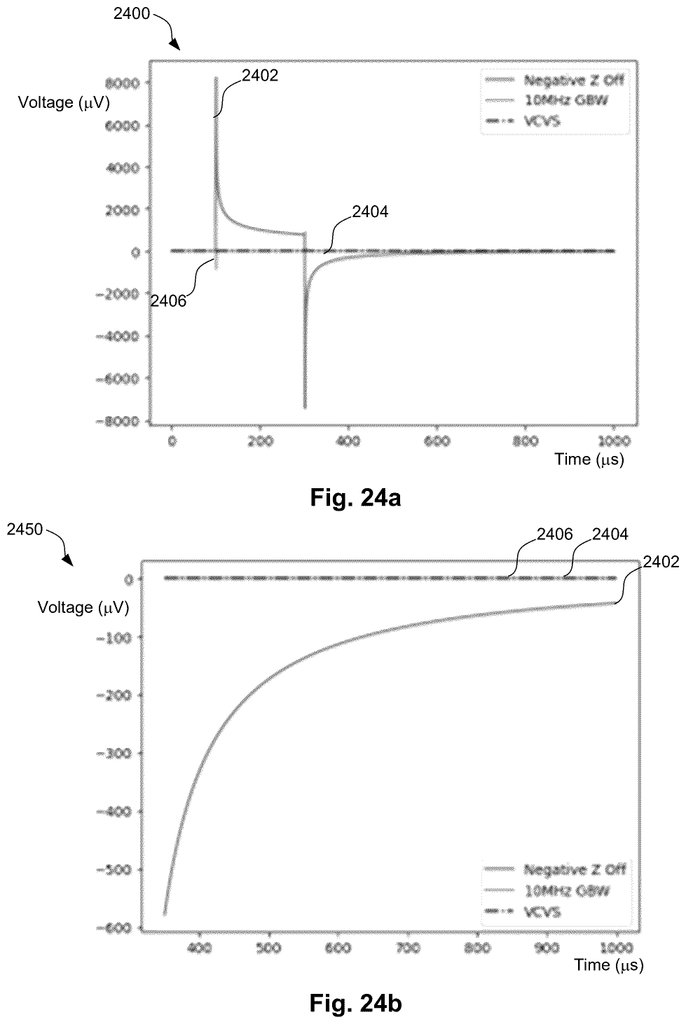

FIG. 24a is a graph 2400 containing plots 2402, 2404, 2406 of voltage across the CPE representing a corresponding measurement electrode of circuit model 2300 during a 1 ms simulation. FIG. 24b illustrates the voltage plots of FIG. 24a commencing 350 μs after the end of the V_{cm} pulse. The plots 2402, 2404, and 2406 show an identical effect of the added negative impedance reducing or eliminating the voltage transient, as observed in the corresponding plots 1602, 1604, and 1606 of FIGS. 16a and 16b. The advantages of using a common Miller amplifier, for example with a star point driver implementation, with a plurality of Miller impedances, over the independent impedance generator implementation of FIG. 15 are that (1) only a single Miller amplifier is needed for both amplifier inputs, and (2) an existing impedance network (the start capacitor network) may be utilised to provide the Miller impedance for each amplifier input. Therefore, no further Miller impedances need be added to achieve the elimination or reduction of mismatch artefact.

### Effect of Attenuation of Differential Signals

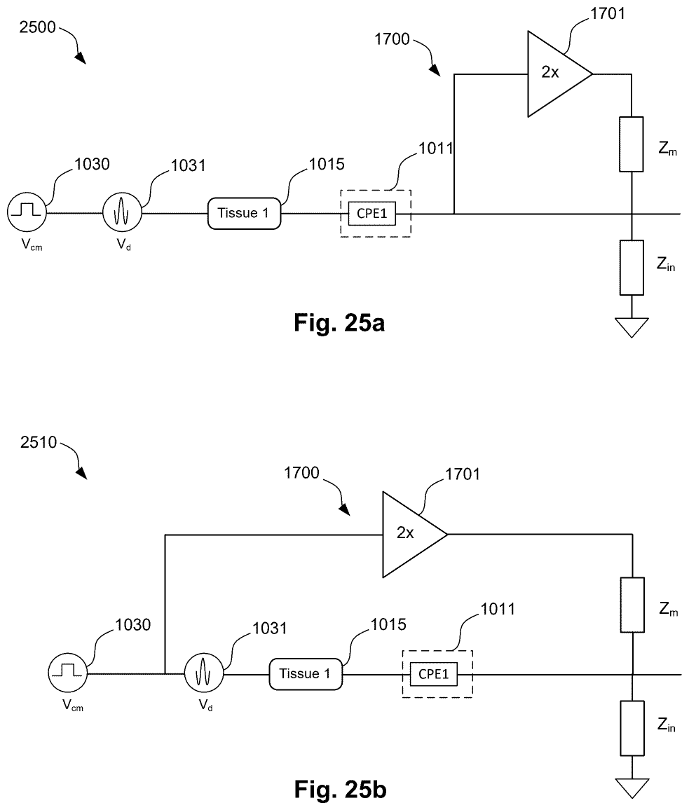

Referring to the common Miller amplifier implementation depicted in FIG. 22, signals that appear differentially at the measurement electrodes 1011 and 1012 (such as ECAPs) may be attenuated. FIGS. 25a and 25b illustrate equivalent circuits 2500 and 2510 of the independent negative Z-generator and common Miller amplifier models with a differential potential source 1031 having value V_{d}. Unlike the independent negative Z-generator model 2500, the common Miller amplifier model 2510 allows a differential source signal with potential V_{d} to drive a current into the input impedance of the measurement amplifier Zᵢₙ, thereby attenuating the potential V_{d} at the measurement amplifier input. This may make the differential potential 1031 harder to detect by the measurement amplifier. However, because its fundamental frequency is relatively low, the attenuation is negligible.

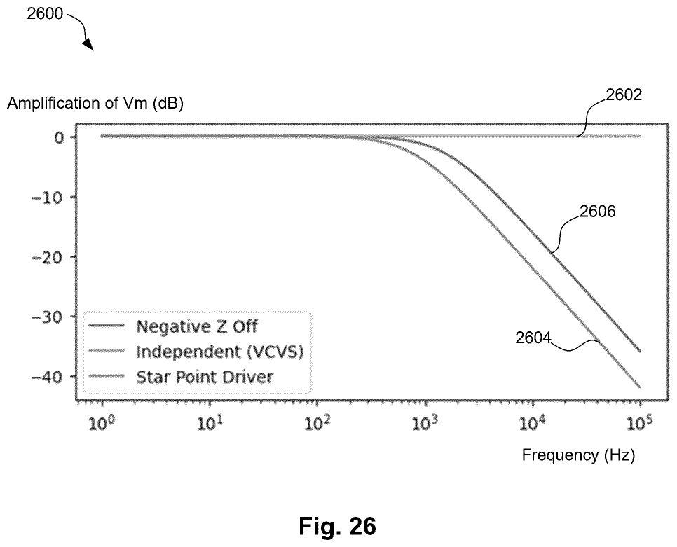

FIG. 26 is a graph 2600 containing plots 2602, 2604, and 2606 of frequency response of the independent negative Z-generator model (VCVS), the common Miller amplifier model (“Star point driver”), and the model without negative impedance (“Negative Z Off”), respectively, to a differential voltage signal V_{d}. The magnitude of the gain of the signal at the measurement amplifier input is shown by each plot. V_{cm} was set to 0V and an ideal VCVS was used for the Miller amplifier of the negative Z-generator circuits.

The graph 2600 was produced by a simulation using a 1 kΩ tissue impedance and a 100 pF input capacitance. In response to the use of an independent negative impedance generator circuit (depicted by the model of FIG. 25a) (plot 2602), no current passes through the CPE as a result of the differential voltage V_{d} and hence there is no attenuation of V_{d}. Without added negative impedance (plot 2606) the 3 db cutoff frequency is 1.6 MHz resulting in attenuation of the V_{d} signal for frequencies above this cutoff value. The use of a common Miller amplifier circuit (as depicted by the model of FIG. 25b) (plot 2604) retains the ability to attenuate V_{d}, where the cutoff frequency drops to 800 kHz. Since V_{cm} is 0V, the Miller capacitor adds to the existing input capacitance. The input capacitance effectively doubles, becoming 200 pF. The ECAP has a fundamental frequency of 1 kHz which is significantly lower than the cutoff of 800 kHz. This shows that the use of a common Miller amplifier circuit (e.g., a star point driver configuration), as connected directly to the common mode potential, will advantageously filter high frequency noise in V_{d} while still permitting the sensing of ECAPs (whose frequency content is mostly well below 800 kHz).

In some examples, it is desirable to perform noise filtering of the common mode potential V_{cm}. This may be performed by adding a pole to the Miller amplifier of the negative Z-generator circuit. For the independent negative Z-generator implementation (e.g., as depicted by the models of FIGS. 14 and 25a), it may be beneficial for the cutoff frequency of each Miller amplifier to be lower than the cutoff frequency of the tissue and the Miller impedance element (e.g., capacitive element Cₘ). This advantageously provides stability to the negative impedance generator circuit despite the presence of both positive and negative feedback.

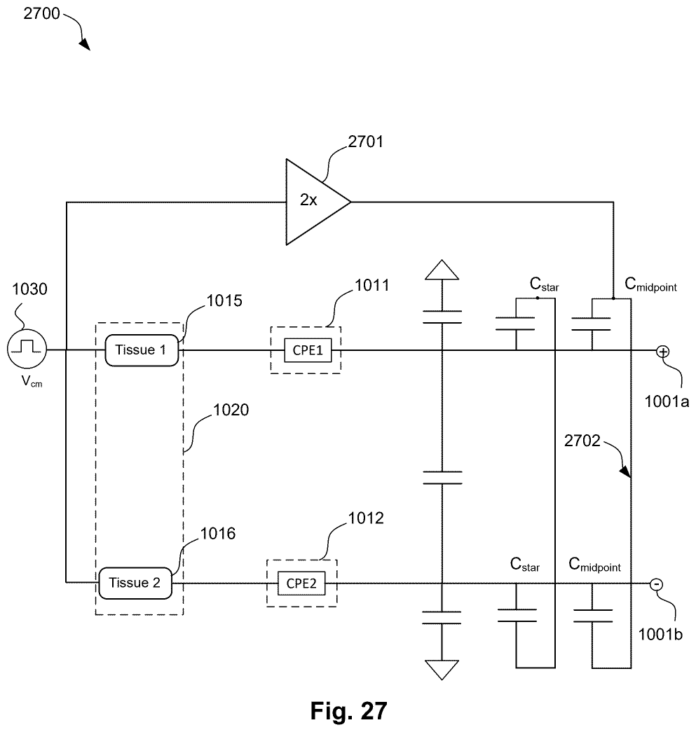

FIG. 27 illustrates an alternative implementation 2700 of the star point driver configuration of FIG. 22, in which additional capacitors C_{midpoint} are arranged in a star configuration 2702 connected between the first input 1001a and the second input 1001b of the measurement amplifier 1001. The Miller amplifier 2701 drives the star point of the star configuration 2702 of additional capacitors, where the additional capacitors are specifically configured as Miller impedances, rather than the star point of the existing filter capacitors Cₛₜₐᵣ. This implementation 2700 advantageously provides flexibility in both the additional capacitance values and location of the additional capacitor network. For example, it may be beneficial to select a capacitance C_{midpoint} that is larger than Cₛₜₐᵣ to reduce the driver supply voltage VDDHV. Advantageously, in the implementation 2700 of FIG. 27 this can be achieved without replacing the plurality of impedances (i.e., the existing network of filter capacitors).

### Method of Neural Response Measurement Using Negative Impedance

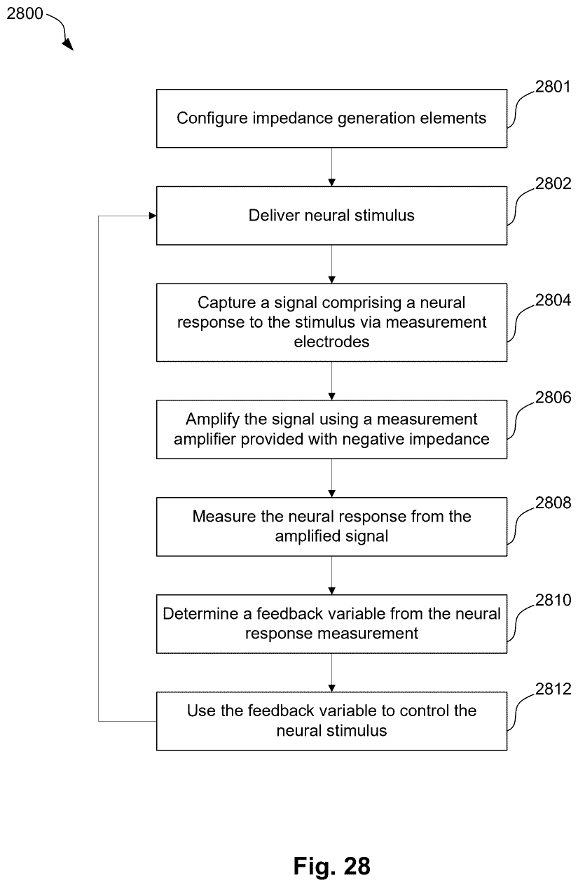

FIG. 28 illustrates a method 2800 for measuring an evoked neural response according to the techniques proposed herein. Method 2800 may be performed by a neural stimulation system operating a neuromodulation device for the purpose of providing neuromodulation therapy, such as for example the neural stimulation system 700 and corresponding neuromodulation device 710 depicted in FIG. 7.

At step 2801, one or more impedance generation elements are optionally configured. The one or more impedance generation elements may include corresponding elements of a negative impedance generator circuit, such as a differential amplifier, a Miller impedance element Zₘ, and a pair of resistors R₁ and R₂ in accordance with the impedance generator circuit 1300 of FIG. 13. In some examples, configuration of the impedance generation elements comprises one or more of: (i) setting the value of the Miller impedance or one or more of the resistances of resistors R₁ and R₂; and (ii) connecting the negative impedance generator circuit comprising the configured elements to at least one of a first input and a second input of a differential measurement amplifier used to perform the method 2800.

In some examples, the measurement amplifier is pre-configured according to step 2801 such that the first input and the second input of the measurement amplifier are each provided with a negative impedance to perform the measurement of an evoked neural response by executing the other steps of method 2800. In some examples, some or all of the activities of the configuration step 2801 take place at another time during the method 2800, such as for example after the capturing of the neural response signal (i.e., step 2804).

At step 2802, a neural stimulus is delivered to neural tissue, the neural stimulus being configured to evoke a neural response from the neural tissue. The neural stimulus is delivered to neural tissue via one or more stimulus electrodes (e.g., electrodes 2 and 4 of electrode array 150, as depicted in FIG. 3), and according to a stimulus intensity parameter. The stimulus intensity parameter is set by a control unit or processor of the neural stimulation system or device.

At step 2804, the neural stimulation system or device captures a signal sensed on the neural tissue subsequent to the delivered stimulus by a first measurement electrode and a second measurement electrode (e.g., electrodes 6 and 8 of the electrode array 150 depicted in FIG. 3), where the sensed signal comprises the neural response evoked by the stimulus.

The sensed signal is amplified using a measurement amplifier at step 2806, and the neural response is subsequently measured from the amplified signal at step 2808. In some examples, the measurement amplifier is an amplifier of measurement circuitry 318 and 128, as depicted in FIGS. 3 and 5, being a differential amplifier connected to the first and second measurement electrodes at respective first and second inputs. At least one of the first input and the second input of the measurement amplifier are provided with a negative impedance according to the techniques proposed herein. In response to the amplification of the sensed signal, the neural response evoked by the delivered neural stimulus is measured, such as for example to determine an intensity of the neural response (e.g., an ECAP amplitude value).

In some examples, the neural stimulation system is a CLNS system (e.g., as depicted in FIG. 5) that performs periodic adjustments to the stimulus intensity parameter to maintain the neural response at or near a target intensity. In such examples, at step 2810 a feedback variable is determined from the measurement of the neural response, and then, at step 2812, the feedback variable is used to control the value of the stimulus intensity parameter for the delivery of further neural stimuli to the tissue (e.g., by repeating step 2802 with an adjusted stimulus intensity parameter).

### Experimental Evaluation

An experimental evaluation was performed of the method 2800 for measuring an evoked neural response using negative impedance generator circuits. A neurostimulation device was connected to an inline CPE-emulating PCB terminated with a 250Ω star load to simulate neural tissue subject to neural stimulation, and from which a neural response is to be measured. The CPE-emulating PCB adds a circuit approximation of a CPE to each stimulation and measurement channel. A general-purpose op-amp was used in a non-inverting configuration to implement a Miller amplifier to drive a 150 pF Miller capacitor to implement the negative impedance generator circuit 1300 of FIG. 13. The Miller amplifier gain was set using a 10K Potentiometer in place of the resistors R1 and R2. The Miller amplifier was powered using a 30V rail voltage (VDDHV).

A set of channels E1-E8 were used to simulate electrodes of an electrode array. Channels E1 and E2 were used for stimulation, at a current of 5 mA. Measurements were made between channels E7 and E8. The CPE on channel E8 was shorted out with a jumper wire to create a large impedance mismatch relative to channel E7. A CPA, as depicted in FIG. 7, was used to configure the stimulator and make artefact measurements.

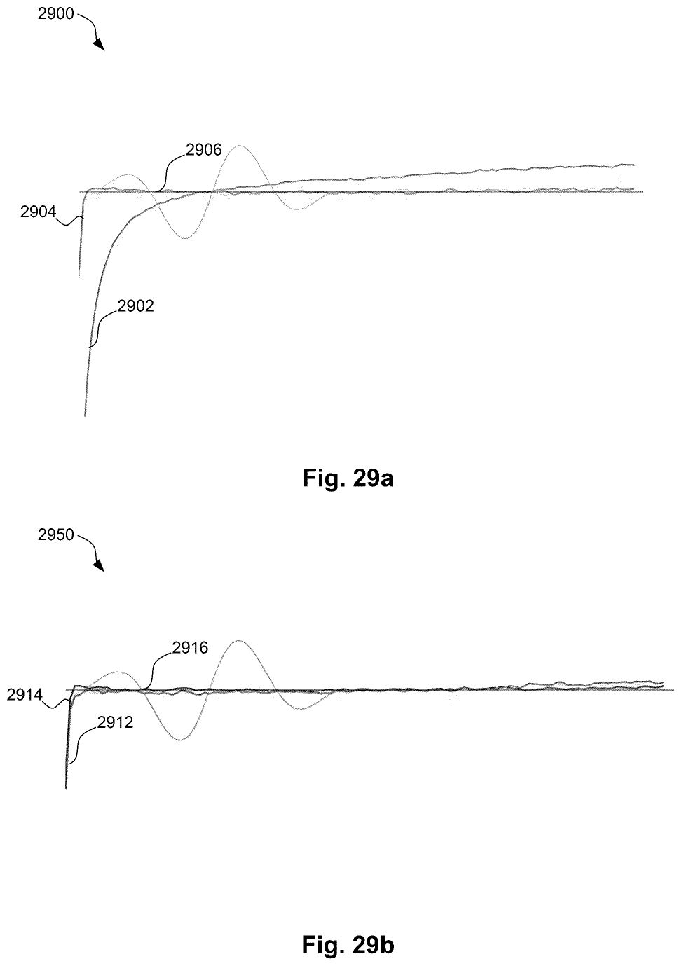

FIG. 29a is a graph 2900 illustrating a first neural response measurement conducted during a simulation without the use of negative impedance at the measurement amplifier inputs. The trace 2902 shows the mismatch artefact on the channels E7-E8. The traces 2904 and 2906 show the artefact measurements for two other measurement electrode pairs with matching CPEs. FIG. 29b is a graph 2950 illustrating a second neural response measurement conducted during the simulation of FIG. 29a with the connection of negative impedance at the inputs of the measurement amplifier (channels E7 and E8). The potentiometer was tuned to zero out the mismatch artefact in the plots 2912, 2914 and 2916, illustrating the effectiveness of the negative impedance when properly configured in reducing or eliminating artefact.

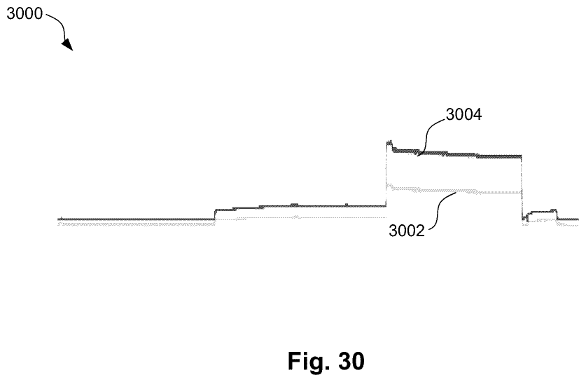

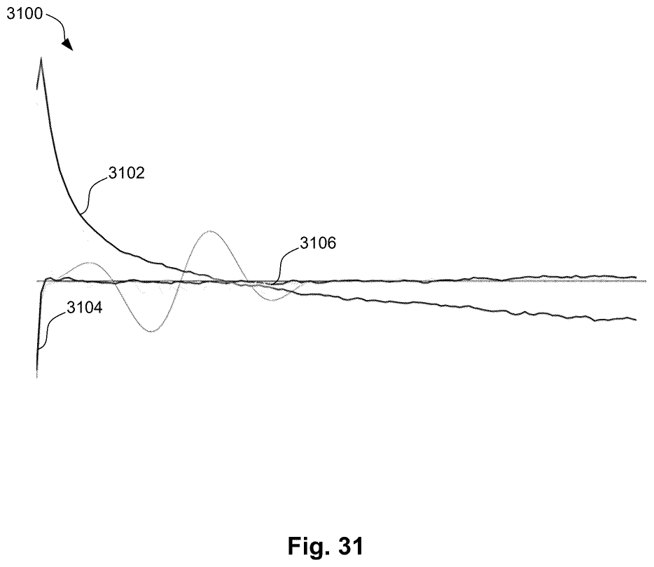

FIG. 30 illustrates a scope capture 3000 of the common mode potential (trace 3002) and the voltage at the output of the Miller amplifier (with gain K=2) of a negative Z-generator circuit (trace 3004) to device ground. The output voltage of the Miller amplifier is approximately twice the common mode potential, as expected.

FIG. 31 is a graph 3100 illustrating a third neural response measurement conducted during the simulation of FIG. 29a, but where the gain of the Miller amplifier was set so that the magnitude of the negative capacitance exceeded the input capacitance of the measurement system. The trace 3102 of graph 3100 shows a sign flip of the mismatch artefact compared to the trace 2902, which further demonstrates the effect of the negative impedance on mismatch artefact. The traces 3104 and 3106 show the artefact measurements for the two other measurement electrode pairs with matching CPEs. These traces are largely the same as the corresponding traces 2904 and 2906 depicted in FIG. 29a.

### Interpretation

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion. The present technology is not limited to any particular programming language or operating system.

### Wireless

In the context of the present disclosure, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of the present disclosure, the term “wired” and its derivatives may be used to describe circuits, devices, systems, methods, techniques, communications channels, etc., that may communicate data through the use of modulated signals propagating through a conductive medium. The term does not imply that the associated devices are coupled by electrically conductive wires.

Wireless communication standards that can be accommodated include IEEE 802.11 wireless LANs and links, Bluetooth, and wireless Ethernet. The technology disclosed herein may be implemented using devices conforming to other network standards and for other applications, including, for example other WLAN standards and other wireless standards such as MICS.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of the present technology.

Furthermore, while some implementations described herein include some, but not other features included in other implementations, combinations of features of different implementations are meant to be within the scope of the present technology, and form different implementations of the present technology, as would be understood by those in the art. For example, in the following claims, any of the claimed implementations can generally be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each value between two particular values is also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicates that different instances of like objects are being referred to, and is not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### Terminology

Throughout the present disclosure, the terms “a” and “an” mean “one or more”, unless expressly specified otherwise.

Throughout the present disclosure, the word “comprise”, or variations such as “comprises” or “comprising”, will be understood to imply the inclusion of a stated element, integer, or step, or group of elements, integers, or steps, but not the exclusion of any other element, integer, or step, or group of elements, integers, or steps.

Throughout the present disclosure, a statement that an element may be “at least one of” or “one or more of” a list of options is to be understood to mean that the element may be any one of the listed options, or may be any combination of two or more of the listed options.

Throughout the present disclosure, the word “or” is to be read inclusively rather than exclusively, except where otherwise indicated.

Neither the title nor any abstract of the present disclosure should be taken as limiting in any way the scope of the claimed invention.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean either that two or more elements are in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology. The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in the present disclosure, including in the claims, are to be understood as falling within the scope of the disclosure of the present disclosure.

### INDUSTRIAL APPLICABILITY

It is apparent from the above that the arrangements described are applicable to the health care industries.

LABEL LIST

| | |
|---|---|
| stimulator | 100 |
| input | 102 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| ground | 130 |
| array | 150 |
| biphasic stimulus pulse | 160 |
| ECAP | 170 |
| nerve | 180 |
| communications channel | 190 |
| external computing device | 192 |
| CLNS system | 300 |
| clinical settings controller | 302 |
| target ECAP controller | 304 |
| box | 308 |
| box | 309 |
| controller | 310 |
| box | 311 |
| stimulator | 312 |
| stimulator | 312A |
| stimulator | 312B |
| stimulator | 312C |
| stimulator | 312D |
| element | 313 |
| measurement circuitry | 318 |
| signal window | 319 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| target ECAP amplitude | 520 |
| ECAP | 600 |
| neural stimulation system | 700 |
| neuromodulation device | 710 |
| remote controller | 720 |
| CST | 730 |
| CI | 740 |
| charger | 750 |
| illustration | 800 |
| first stimulus pulse | 810 |
| inter-stimulus interval | 815 |
| stimulus pulse | 820 |
| stimulus pulse | 830 |
| stimulus pulse | 840 |
| stimulus pulse | 850 |
| ECAP | 860 |
| multi - stimset CLNS system | 900 |
| circuit | 1000 |
| amplifier | 1001 |
| input | 1001a |
| input | 1001b |
| Output | 1001c |
| differential potential source | 1031 |
| graph | 1060 |
| CPE voltage trace | 1062 |
| waveform | 1064 |
| electrical circuitry | 1100 |
| model | 1100a |
| model | 1100b |
| equivalent impedance | 1111 |
| equivalent impedance | 1112 |
| first set | 1113 |
| second set | 1114 |
| circuit | 1200 |
| Miller amplifier | 1201 |
| input | 1202 |
| output | 1204 |
| circuit | 1210 |
| circuit | 1300 |
| amplifier | 1301 |
| non - inverting input | 1301a |
| inverting input | 1301b |
| circuit model | 1400 |
| negative Z - generator | 1402 |
| negative Z - generator | 1404 |
| impedance element | 1406 |
| impedance element | 1408 |
| circuit model | 1500 |
| graph | 1600 |
| plot | 1602 |
| plot | 1604 |
| plot | 1606 |
| circuit | 1700 |
| Miller amplifier | 1701 |
| graph | 1800 |
| plot | 1802 |
| plot | 1804 |
| plot | 1806 |
| graph | 1900 |
| graph | 2000 |
| model | 2100 |
| Miller amplifier | 2101 |
| negative Z - generator | 2102 |
| circuit model | 2110 |
| circuit model | 2200 |
| circuit model | 2300 |
| graph | 2400 |
| plot | 2402 |
| plot | 2404 |
| plot | 2406 |
| model | 2500 |
| model | 2510 |
| graph | 2600 |
| plot | 2602 |
| plot | 2604 |
| plot | 2606 |
| implementation | 2700 |
| Miller amplifier | 2701 |
| star configuration | 2702 |
| method | 2800 |
| step | 2801 |
| step | 2802 |
| step | 2804 |
| step | 2806 |
| step | 2808 |
| step | 2810 |
| step | 2812 |
| graph | 2900 |
| trace | 2902 |
| trace | 2904 |
| trace | 2906 |
| plot | 2912 |
| plot | 2914 |
| plot | 2916 |
| graph | 2950 |
| scope capture | 3000 |
| trace | 3002 |
| trace | 3004 |
| graph | 3100 |
| trace | 3102 |
| trace | 3104 |
| trace | 3106 |

1. An implantable device for measuring an evoked neural response, the implantable device comprising:
a stimulus source configured to deliver neural stimuli via one or more stimulus electrodes to neural tissue, the neural stimuli being configured to evoke a neural response from the neural tissue;
a measurement amplifier configured to amplify a signal sensed between a first input of the measurement amplifier by a first measurement electrode and a second input of the measurement amplifier by a second measurement electrode subsequent to a provided neural stimulus, the sensed signal comprising the evoked neural response;
a control unit configured to:
control the stimulus source to deliver a neural stimulus; and
measure the evoked neural response of the amplified sensed signal; and
one or more impedance elements configured to provide a negative impedance to at least one of the first and second inputs of the measurement amplifier.

2. The implantable device of claim 1, wherein the one or more impedance elements comprise:
a first set of impedance elements connected to the first input of the measurement amplifier and the first measurement electrode; and a second set of impedance elements connected to the second input of the measurement amplifier and the second measurement electrode.

3. The implantable device of claim 2, wherein the first and second sets of impedance elements provide respective first and second negative impedances to the first and second inputs in parallel to respective input impedances of the measurement amplifier at the respective inputs.

4. The implantable device of claim 3, wherein the value of the negative impedance provided to each of the first and second inputs of the measurement amplifier is equal, or substantially equal, to the negative of the value of the input impedance of the measurement amplifier at the corresponding input.

5. The implantable device of claim 3, wherein the value of the negative impedance provided to each of the first and second inputs of the measurement amplifier increases the value of a total impedance at the corresponding input of the measurement amplifier to at least a threshold impedance value.

6. The implantable device of claim 5, wherein the threshold impedance value is sufficiently large to suppress a transient voltage generated at an electrode-tissue interface of a corresponding one of the measurement electrodes as a result of the neural stimulus to a degree that enables measurement of the evoked neural response.

7. The implantable device of claim 1, wherein the one or more impedance elements comprise one or more respective negative impedance generator circuits.

8. The implantable device of claim 7, wherein each negative impedance generator circuit comprises a Miller amplifier having a Miller impedance element connected across its input and its output, wherein a gain of the Miller amplifier is such that an effective input impedance to ground at the input of the Miller amplifier provides the negative impedance.

9. The implantable device of claim 8, wherein the input of each Miller amplifier is connected to one of the first and second measurement electrodes.

10. The implantable device of claim 8, wherein the input of at least one Miller amplifier is connected to the neural tissue.

11. The implantable device of claim 8, wherein the value of the negative impedance provided by each negative impedance generator circuit is adjustable by adjusting a gain of the corresponding Miller amplifier.

12. The implantable device of claim 8, wherein the value of each Miller impedance element is set to approximate, or be equal to, a corresponding input impedance of the measurement amplifier.

13. The implantable device of claim 12, wherein each Miller impedance element is substantially capacitive with the capacitance value equal to a total input capacitance to ground at the corresponding input of the measurement amplifier.

14. The implantable device of claim 7 wherein the first input of the measurement amplifier is connected to a first negative impedance generator circuit and the second input of the measurement amplifier is connected to a second negative impedance generator circuit.

15. The implantable device of claim 14, wherein the first and second negative impedance generator circuits are independent circuits.

16. The implantable device of claim 15, wherein each negative impedance generator circuit comprises a Miller amplifier having a Miller impedance element connected across its input and its output, and wherein each Miller amplifier has a pole with a cutoff frequency less than a frequency of the neural tissue and the Miller impedance element.

17. The implantable device of claim 14, wherein the first and second negative impedance generator circuits share a common Miller amplifier.

18. The implantable device of claim 17, wherein the common Miller amplifier drives a star point of a plurality of impedances arranged in a star configuration across the first input and the second input of the measurement amplifier.

19. The implantable device of claim 18, wherein each impedance of the plurality of impedances provides a separate Miller impedance element to the common Miller amplifier.

20. The implantable device of claim 18, wherein the plurality of impedances comprises a plurality of filter capacitors each having a capacitance of at least 100 pF.

21. A method for measuring an evoked neural response, the method comprising:
delivering a neural stimulus via one or more stimulus electrodes to neural tissue, the neural stimulus being configured to evoke a neural response from the neural tissue, and the neural stimulus being delivered according to a stimulus intensity parameter;
capturing a signal sensed on the neural tissue by a first measurement electrode and a second measurement electrode, the sensed signal comprising the evoked neural response;
using a measurement amplifier to amplify the sensed signal, the measurement amplifier having a first input connected to the first measurement electrode and a second input connected to the second measurement electrode; and
measuring the neural response evoked by the delivered neural stimulus, wherein at least one of the first input and the second input of the measurement amplifier are provided with a negative impedance.

22. The method of claim 21, further comprising configuring one or more impedance elements to provide at least one of the first input and the second input of the measurement amplifier with a negative impedance.

23. The method of claim 22, wherein the negative impedance provided to the at least one of the first input and the second input of the measurement amplifier is provided in parallel to an input impedance of the measurement amplifier at the at least one input.

24. The method of claim 23, wherein configuring one or more impedance elements comprises setting the value of the negative impedance provided to the at least one of the first and second inputs of the measurement amplifier to be equal, or substantially equal, to the negative of the value of the input impedance of the measurement amplifier at the at least one input.

25. The method of claim 23, wherein configuring one or more impedance elements comprises setting the value of the negative impedance provided to the at least one of the first and second inputs of the measurement amplifier to increase the value of a total input impedance of the measurement amplifier at the at least one input to at least a threshold impedance value.

26. The method of claim 25, wherein the threshold impedance value is sufficiently large to suppress a transient voltage generated at an electrode-tissue interface of the at least one corresponding measurement electrode as a result of the neural stimulus to a degree that enables measurement of the evoked neural response.

27. The method of claim 21, wherein the one or more negative impedances are generated by one or more respective negative impedance generator circuits.

28. The method of claim 27, wherein each negative impedance generator circuit comprises a Miller amplifier having a Miller impedance element connected across its input and its output, wherein a gain of the Miller amplifier is such that the effective input impedance to ground at the input of the Miller amplifier provides the negative impedance.

29. The method of claim 28, further comprising adjusting the value of the negative impedance provided by each negative impedance generator circuit by adjusting a gain of the corresponding Miller amplifier.

30. The method of claim 28, further comprising setting the value of each Miller impedance element to approximate, or be equal to, a corresponding input impedance of the measurement amplifier.

31. The method of claim 30, wherein each Miller impedance element is substantially capacitive with the capacitance value equal to a total input capacitance to ground at the corresponding input of the measurement amplifier.

32. The method of claim 27, wherein the first input of the measurement amplifier is connected to a first negative impedance generator circuit and the second input of the measurement amplifier is connected to a second negative impedance generator circuit.

33. The method of claim 32, wherein the first and second negative impedance generator circuits are independent circuits.

34. The method of claim 33, wherein each negative impedance generator circuit comprises a Miller amplifier having a Miller impedance element connected across its input and its output, and wherein each Miller amplifier has a pole with a cutoff frequency less than a frequency of the neural tissue and the Miller impedance element.

35. The method of claim 32, wherein the first and second negative impedance generator circuits share a common Miller amplifier.

36. The method of claim 35, wherein the common Miller amplifier drives a star point of a plurality of impedances arranged in a star configuration across the first input and the second input of the measurement amplifier.

37. The method of claim 36, wherein each impedance of the plurality of impedances provides a separate Miller impedance element to the common Miller amplifier.

38. The method of claim 36, wherein the plurality of impedances comprises a plurality of filter capacitors each having a capacitance of at least 100 pF.

39. The method of claim 21, further comprising:
computing, from an intensity of the measured evoked neural response, a feedback variable; and
completing a feedback loop by using the computed feedback variable to control the stimulus intensity parameter so as to maintain the feedback variable at a target value.

40. A neural stimulation system comprising:
a neural stimulation device for controllably delivering neural stimuli to neural tissue, the device comprising:
a control unit configured to:
control a stimulus source to deliver a neural stimulus via one or more stimulus electrodes to neural tissue, the neural stimulus being configured to evoke a neural response from the neural tissue; and
use a measurement amplifier having first and second inputs connected to corresponding first and second measurement electrodes to amplify a signal sensed by the first and second measurement electrodes subsequent to the delivered neural stimulus, the sensed signal comprising the evoked neural response, wherein at least one of the first and second inputs of the measurement amplifier are provided with a negative impedance; and
a processor configured to measure the neural response evoked by the delivered neural stimulus based on the amplified sensed signal.
